(19) United States
(12) Patent Application Publication
(10) Pub. No.: US 2007/0122830 A1
(43) Pub. Date: May 31, 2007

(54) Prohibitin-directed diagnostics and therapeutics for cancer and chemotherapeutic drug resistance

(75) Inventors: Elias Georges, Laval (CA); Panagiotis Prinos, Montreal (CA)

Correspondence Address: WILMER CUTLER PICKERING HALE AND DORR LLP, 60 STATE STREET, BOSTON, MA, 02109, US

(73) Assignee: Aurelium BioPharma Inc., Montreal (CA)

(21) Appl. No.: 11/595,560

(22) Filed: Nov. 9, 2006

Related U.S. Application Data
(60) Provisional application No. 60/735,478, filed on Nov. 10, 2005.

(51) Int. Cl. C12Q 1/68; G01N 33/574

(52) U.S. Cl. 435/6; 435/7.23

(57) ABSTRACT

Disclosed are methods for treating neoplastic cells, including reversing or preventing chemotherapeutic drug resistance, by increasing the sensitivity of the neoplastic cells to a chemotherapeutic drug. In addition, methods are further disclosed for diagnosing chemotherapeutic drug resistance in neoplastic cells by detecting an increase in the expression of prohibitin in such neoplastic cells as compared to the level of expression of prohibitin protein in a non-MDR neoplastic cell.

This Application claims the benefit of priority to U.S. Provisional Application No. 60/735,478, filed Nov. 10, 2005, the specification of which is incorporated by reference in its entirety.

### FIELD OF THE INVENTION

This invention relates to the field of cancer. In particular, this invention relates to the detection, diagnosis, and treatment of neoplastic cells, and more specifically to the detection and treatment of chemotherapeutic drug-resistant neoplastic cells.

### BACKGROUND OF THE INVENTION

Cancer is often treated with chemotherapeutics such as cytotoxic drugs. In order to kill the cancer or diseased cells, the drug(s) must enter the cells and reach an effective dose so as to interfere with essential biochemical pathways. Generally, chemotherapeutic drugs disrupt cellular mechanisms such as DNA replication and osmotic control to bring about apoptosis of the cell. Unfortunately, although chemotherapeutic drugs are effective at killing neoplastic cells, they also tend to be indiscriminate killers of other cells in the subject, targeting healthy and neoplastic cells with equal efficacy. As a result, chemotherapy treatments are generally provided to the subject for as short a period as possible to limit the detrimental effects of the drug on the subject.

Chemotherapy drug treatments may be limited by the inherent sensitivity of the cancer cell to the drug being used in the treatment, which can vary from cancer type to cancer type. In some cases, a treatment regime lasting for a long duration may be required due to the relative insensitivity of the cells to the treatment, increasing the patient's exposure to drugs that are toxic to both normal and cancer cells. However, as described above, prolonged treatment periods may increase the likelihood that the patient will suffer from detrimental side effects attributable to the treatment regime. Common side effects include neutropenia, anemia, thrombocytopenia, nausea, hair loss, organ and tissue damage, and infections. Although most side effects are normally tolerable compared to the symptoms of the disease, chemotherapeutic side effects can, in some instances, lead to cessation of the treatment regime or death. As a result of these potentialities, many patients suffer significant emotional and physiological consequences associated with the treatment regime.

In addition to the inherent sensitivity of particular cancer cell types to chemotherapeutic drugs, cancer cells may evade being killed by the drug through the development of resistance to it (termed “drug resistance”). Moreover, in some cases, cancer cells (also called “tumor” cells or “neoplastic” cells) develop resistance to a broad spectrum of drugs, including drugs that were not originally used for treatment. This phenomenon is termed “chemotherapeutic drug resistance.” Chemotherapeutic drug resistance arises through different mechanisms, and each mechanism is associated with a different biological marker or group of markers that may be clinically useful for detecting and diagnosing the presence of drug resistance.

The emergence of the chemotherapeutic drug resistance, and also the multi-drug resistance (“MDR”) phenotype is the major cause of failure in the treatment of cancer (see, e.g., Davies (1994) Science 264: 375-382; Poole (2001) Cur. Opin. Microbiol. 4: 500-5008). The chemotherapeutic drug resistance phenotype can arise in response to a broad spectrum of functionally distinct drugs, thereby limiting available treatment options. The development of chemotherapeutic drug-resistant cancer cells is the principal reason for treatment failure in cancer patients (see, e.g., Gottesman (2000) Ann. Rev. Med. 53: 615-627).

The sensitivity of cancer cells to a particular drug is normally associated with genes that are utilized in drug metabolism or transport. For example, the classic MDR phenotype involves alterations in a gene for P-glycoprotein, a plasma membrane protein that actively transports drugs out of the cell (see, e.g., Volm et al., (1993) Cancer 71: 3981-3987). In addition, there are many other genes that affect the sensitivity of a cancer cell to a particular drug or class of drugs (see, e.g., Di Nicolantonio et al., (2005) BMC Cancer. 5(1): 78). Thus, it is clear that chemotherapeutic drug sensitivity and multi-drug resistance are multi-factorial traits.

One such factor contributing to the development of cancer, and potentially to multi-drug resistance, is the generation of mutations in cell cycle control genes (see, e.g., Ludwig et al. (2005) Cancer. 104(9): 1794-1807). Cell cycle control genes are important regulators of apoptosis, cell growth, and cell differentiation (see, e.g., Ludwig et al. (2005) Cancer. 104(9): 1794-1807). Mutations in such genes, and genes that control their expression, have been associated with a wide variety of cancers in most tissues. For example, mutations in the tumor suppressor gene p53 have been linked with most cancers studied to date (see, e.g., Wesierska-Gadek et al. (2005) Cell Mol. Biol. Lett. 10(3): 439-53).

Recently, the putative tumor suppressor gene, prohibitin, has been implicated in the regulation of cell cycle progression and apoptosis in cell lines (see, e.g., Mishra et al. (2005) Trends Mol. Med. 11(4): 192-197). Prohibitin appears to be involved primarily in preventing cells from progressing through the cell cycle (Fraser et al. (2003) Rep. Biol. Endrocrin. 1: 66-79). For instance, prohibitin has been observed in normal ovarian cells, where it is upregulated during progression through apoptosis (see, e.g., Fraser et al. (2003) Rep. Biol. Endrocrin. 1: 66-79). It has also been shown to induce growth arrest in mammalian fibroblasts and HeLa cells (see, e.g., Fraser et al. (2003) Rep. Biol. Endrocrin. 1: 66-79). However, prohibitin also has been shown to be an anti-apoptotic gene by regulating anti-apoptotic pathways in certain cell lines (see, e.g., Fraser et al. (2003) Rep. Biol. Endrocrin. 1: 66-79). Therefore, prohibitin appears to be associated with several different, and antagonistic, cellular functions.

There remains a need for methods and compositions that detect, treat, and prevent cancer. Furthermore, there remains a need in both humans and animals for detecting, treating, preventing, and reversing the development of both classical and atypical MDR phenotypes in cancer cells and non-cancerous damaged cells, regardless of how the MDR arises (e.g., naturally occurring or drug-induced). In addition, the ability to identify, and to make use of, reagents that target cancer cells and MDR cells have clinical potential for improvements in the diagnosis of chemotherapeutic drug resistance. Such reagents also have the clinical potential to improve treatments for cancer, including chemotherapeutic drug resistant cancers. Also, there remains a need for increasing the sensitivity of cancer cells to chemotherapeutic drugs in order to shorten the time period of chemotherapeutic treatment in both humans and animals. By shortening the time period of chemotherapeutic treatment and allowing physicians to make appropriate chemotherapy treatment choices, there is a potential for significant improvements in treatment of neoplasms.

### SUMMARY OF THE INVENTION

The present invention is based, in part, upon the discovery that prohibitin, a calcium binding protein localized to the endoplasmic reticulum and the Golgi complex of the cell, is expressed at higher levels in neoplastic cells that have developed chemotherapeutic drug resistance. This discovery has been utilized to provide the present invention that, in part, is directed to therapeutic methods and compositions for treating neoplastic cells, including neoplastic cells that have developed chemotherapeutic drug resistance, through the use of targeting agents specific for prohibitin. The invention, in part, also provides a method that uses targeting agents specific for prohibitin to detect and diagnose chemotherapeutic drug resistance in neoplastic cells in a subject.

Accordingly, in one aspect, the invention provides a method of diagnosing chemotherapeutic drug resistance in a neoplastic cell that is not a cervical squamous cell carcinoma or is not derived therefrom. The method comprises detecting a level of prohibitin expressed in a potentially chemotherapeutic drug-resistant neoplastic cell sample, by contacting that cell sample with a targeting agent specific for prohibitin. Then, a level of prohibitin expressed in a non-resistant neoplastic control cell sample of the same tissue type as the neoplastic cell sample is detected, by contacting the control cell sample with a prohibitin-specific targeting agent. The level of expressed prohibitin in the potentially chemotherapeutic drug resistant neoplastic cell sample is then compared to the level of detected prohibitin in the non-resistant control neoplastic cell. Chemotherapeutic drug-resistance is indicated in the neoplastic cell sample if the level of prohibitin expressed is greater than the level of prohibitin expressed in the non-resistant neoplastic control cell sample.

In certain embodiments, the detection steps comprise isolating a cytoplasmic sample from the neoplastic cell sample and the non-resistant neoplastic control cell sample. In other embodiments, detecting the level of expressed prohibitin in the cell samples comprises contacting the cell samples with a prohibitin targeting agent such as nucleic acids, antibodies, or prohibitin-binding fragments of antibodies. In particular embodiments, the prohibitin-targeting agent comprises an anti-prohibitin antibody or a prohibitin-binding fragment thereof. In some embodiments, the level of antibody bound to prohibitin is detected by immunofluorescence, radiolabel, or chemiluminescence.

In further embodiments, the prohibitin-specific targeting agent comprises a nucleic acid probe complementary to prohibitin mRNA. In certain embodiments, the nucleic acid probe is selected from the group consisting of RNA, DNA, RNA-DNA hybrids, and siRNA. In some embodiments, the probe is an antisense oligonucleotide or ribozyme. In some embodiments, the level of nucleic acid probe hybridized to prohibitin mRNA is detected with a label such as one selected from the group consisting of fluorophores, chemical dyes, radiolabels, chemiluminescent compounds, colorimetric enzymatic reactions, chemiluminescent enzymatic reactions, magnetic compounds, and paramagnetic compounds.

In certain embodiments, the neoplastic control cell sample is lung carcinoma, lung adenocarcinoma, colon carcinoma, ovarian carcinoma, or ovarian adenocarcinoma. In some embodiments, the potentially chemotherapeutic drug resistant neoplastic cell sample to be tested comprises a breast adenocarcinoma. In particular embodiments, the neoplastic cell sample to be tested is isolated from a mammal or a human. In certain embodiments, the potentially chemotherapeutic drug-resistant neoplastic cell sample is isolated from a tissue such as breast, skin, lymphatic, prostate, bone, blood, brain, liver, thymus, kidney, lung, or ovary.

In another aspect, the invention provides a method of treating a neoplasm in a patient that is not a cervical squamous cell carcinoma, in a patient. The method comprises administering an effective amount of a prohibitin-targeting agent to the patient, the targeting agent binding to prohibitin expressed by the neoplasm. The method further entails administering to the patient an effective amount of a chemotherapeutic drug. The prohibitin-targeting agent, when bound to the neoplasm, increases the sensitivity of the neoplasm to the chemotherapeutic drug. The prohibitin-targeting agent and the chemotherapeutic drug being administered simultaneously in certain embodiments.

In some embodiments, the prohibitin-targeting agent bound to the neoplasm is internalized by the neoplastic cell. In certain embodiments, the targeting agent is selected from the group consisting of nucleic acids and antibodies or prohibitin-binding fragments thereof. In some embodiments, the prohibitin-targeting agent comprises a liposome. In particular embodiments, the liposome comprises a neoplastic cell-targeting agent on its surface. In still further embodiments, the prohibitin-targeting agent is selected from the group consisting of ligands, nucleic acids, synthetic small molecules, peptidomimetic compounds, inhibitors, peptides, proteins, and antibodies. In particular embodiments, the prohibitin-targeting agent comprises a nucleic acid. In more particular embodiments, the nucleic acid is complementary to a prohibitin mRNA. In still more particular embodiments, the nucleic acid is selected from the group consisting of RNA, DNA, RNA-DNA hybrids, and siRNA. In yet more particular embodiments, the siRNA comprises 19 contiguous nucleotides of SEQ ID NO: 2 or it comprises 25 contiguous nucleotides of SEQ ID NO: 4.

In other embodiments, the prohibitin-targeting agent comprises an antibody or prohibitin-binding fragment thereof. In particular embodiments, the neoplastic cell-targeting agent comprises an antibody, or antigen-binding fragment thereof, specific for a cell marker selected from the group consisting of multidrug resistance protein 1, BRCP, p53, vimentin, α-enolase, nucleophosmin, and HSC70.

In some embodiments, the prohibitin-targeting agent is administered to the patient by injection at the site of the neoplasm. In other embodiments, the prohibitin-targeting agent is administered to the patient by surgical introduction at the site of the neoplasm. In still other embodiments, the prohibitin-targeting agent is administered to the patient by inhalation of an aerosol or vapor.

In certain embodiments, the neoplasm to be treated is chemotherapeutic drug-resistant. In particular embodiments, the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristin, Vinorelbine, and combinations thereof.

In another aspect, the invention provides a kit for detecting chemotherapeutic drug resistance in a neoplastic cell sample. The kit comprises a targeting agent for the detection of prohibitin and a probe for the detection of chemotherapeutic drug resistance, the probe being specific for a marker selected from the group consisting of multidrug resistance protein 1, BRCP, p53, vimentin, α-enolase, nucleophosmin, and HSC70. The kit also provides at least one detection means for identifying binding of probe to a target.

In some embodiments, the targeting agent is selected from the group consisting of nucleic acids and antibodies or prohibitin-binding fragments thereof. In certain embodiments, the targeting agent is a nucleic acid that is complementary to mRNA encoding prohibitin. In particular embodiments, the nucleic acid is selected from the group consisting of RNA, DNA, RNA-DNA hybrids, and siRNA.

In other embodiments, the first probe is a prohibitin-specific antibody or prohibitin-binding fragment thereof. In some embodiments, the probe comprises a nucleic acid complementary to an mRNA encoding multidrug resistance protein 1, BRCP, p53, vimentin, α-enolase, nucleophosmin, or HSC70. In certain embodiments, the nucleic acid probe is selected from the group consisting of RNA, DNA, RNA-DNA hybrids, and siRNA.

In other embodiments, the probe comprises an antibody or prohibitin-binding fragment thereof. In certain embodiments, the detection means is selected from the group consisting of fluorophores, chemical dyes, radiolabels, chemiluminescent compounds, colorimetric enzymatic reactions, chemiluminescent enzymatic reactions, magnetic compounds, and paramagnetic compounds.

In yet another aspect, the invention provides a pharmaceutical formulation for treating a neoplasm. The pharmaceutical formulation comprises a prohibitin-targeting agent, a chemotherapeutic drug, and a pharmaceutically acceptable carrier.

In some embodiments, the prohibitin-specific targeting agent is a nucleic acid, an antibody, or a prohibitin-binding fragment of an antibody. In particular embodiments, the prohibitin-targeting agent is a nucleic acid, such as RNA, DNA, RNA-DNA hybrids, and siRNA. In still more particular embodiments, the prohibitin-targeting agent is a siRNA. In one embodiment, the siRNA has a GC content of at least 40%. In certain embodiments, the siRNA comprises 19 contiguous nucleotides of SEQ ID NO: 1.

In other embodiments, the prohibitin-targeting agent comprises an antibody or prohibitin-binding fragment thereof. In some embodiments, the prohibitin-targeting agent comprises a liposome. In certain embodiments, the liposome comprises a neoplastic cell-targeting agent on its surface.

In specific embodiments, the neoplastic cell-targeting agent is an antibody, or binding fragment thereof. In other embodiments, the neoplastic cell-targeting agent binds to a neoplastic cell marker selected from the group consisting of multidrug resistance protein 1, BRCP, p53, vimentin, α-enolase, nucleophosmin, and HSC70. In further embodiments, the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristin, Vinorelbine, and combinations thereof.

In yet another aspect, the invention provides a method of diagnosing adriamycin resistance in an ovarian neoplastic cell. The method comprises detecting a level of prohibitin expressed in a potentially adriamycin-resistant ovarian cell sample by contacting the cell sample with an antibody specific for prohibitin. The method also comprises the detection of a level of prohibitin expressed in a non-resistant ovarian control cell sample by contacting the cell sample with a prohibitin-specific antibody. The method further comprises comparing the level of expressed prohibitin in the potentially adriamycin-resistant ovarian cell sample to a level of expressed prohibitin in the non-resistant ovarian control cell sample. The potentially adriamycin-resistant ovarian cell sample, which is not a cervical squamous cell carcinoma or not being derived therefrom, is adriamycin-resistant if the level of prohibitin expressed therein is greater than the level of prohibitin expressed in the non-resistant ovarian control cell sample.

### BRIEF DESCRIPTION OF THE FIGURES

The foregoing and other objects of the present invention, the various features thereof, as well as the invention itself may be more fully understood from the following description, when read together with the accompanying drawings in which:

FIG. 1A is a photographic representation of an immunoblot probed with anti-prohibitin antibody that shows the level of expression of prohibitin in drug-resistant and drug-sensitive breast cancer cell line extracts.

FIG. 1B is a photographic representation of an immunoblot probed with anti-prohibitin antibody that shows the level of expression of prohibitin in drug-resistant and drug-sensitive ovarian cancer cell line extracts.

FIG. 1C is a photographic representation of an immunoblot probed with anti-prohibitin antibody that shows the level of expression of prohibitin in HS578T cell extracts, BT549 cell extracts, HeLa cell extracts, and doxorubicin-resistant and doxorubicin-sensitive H69 cell extracts.

FIG. 2 is a photographic representation of an immunoblot probed with anti-prohibitin antibody that shows the level of expression of prohibitin in cell extracts from MDA cells treated with mock siRNA or prohibitin siRNA.

FIG. 3 is a photographic representation of an immunoblot probed with anti-prohibitin antibody that shows the level of expression of prohibitin in cell extracts from MDA cells treated with mock siRNA or prohibitin siRNA.

FIG. 4 is a photographic representation of an immunoblot probed with anti-prohibitin antibody that shows the level of expression of prohibitin in cell extracts from MDA cells treated with Cy3 siRNA or prohibitin siRNA.

FIG. 5 is a photographic representation of an immunoblot probed with anti-prohibitin antibody that shows the level of expression of prohibitin in cell extracts from MDA cells treated with mock siRNA, Cy3 siRNA, B23 siRNA, or prohibitin siRNA.

FIG. 6 is a photographic representation of an immunoblot probed with anti-prohibitin antibody that shows the level of expression of prohibitin in cell extracts from MCF-7 cells treated with mock siRNA or prohibitin siRNA.

FIG. 7 is a photographic representation of an immunoblot probed with anti-prohibitin antibody that shows the level of expression of prohibitin in cell extracts from SKOV3 cells treated with mock siRNA or prohibitin siRNA.

FIG. 8 is a photographic representation of an immunoblot probed with anti-prohibitin antibody that shows the level of expression of prohibitin in cell extracts from SKOV3 cells treated with mock siRNA or prohibitin siRNA.

FIG. 9A is a graphic representation of the results of an MTT cytotoxicity assay that shows the viability of prohibitin siRNA transfected MDA cells treated with adriamycin compared to mock transfected MDA controls.

FIG. 9B is a graphic representation of the results of an MTT cytotoxicity assay that shows the viability of prohibitin siRNA transfected MDA cells treated with cisplatinum compared to mock transfected MDA controls.

FIG. 9C is a graphic representation of the results of an MTT cytotoxicity assay that shows the viability of prohibitin siRNA transfected MDA cells treated with taxol compared to mock transfected MDA controls.

FIG. 9D is a graphic representation of the results of an MTT cytotoxicity assay that shows the viability of prohibitin siRNA transfected MDA cells treated with etoposide compared to mock transfected MDA controls.

FIG. 9E is a graphic representation of the results of an MTT cytotoxicity assay that shows the viability of prohibitin siRNA transfected MDA cells treated with thiotepa compared to mock transfected MDA controls.

FIG. 9F is a graphic representation of the results of an MTT cytotoxicity assay that shows the viability of prohibitin siRNA transfected MDA cells treated with melphalan compared to mock transfected MDA controls.

FIG. 9G is a graphic representation of the results of an MTT cytotoxicity assay that shows the viability of prohibitin siRNA transfected MDA cells treated with mitoxantrone compared to mock transfected MDA controls.

FIG. 9H is a graphic representation of the results of an MTT cytotoxicity assay that shows the viability of prohibitin siRNA transfected MDA cells treated with vincristin compared to mock transfected MDA controls.

FIG. 10A is a graphic representation of the results of an MTT cytotoxicity assay that shows the viability of prohibitin siRNA transfected MDA cells treated with adriamycin compared to mock transfected MDA controls.

FIG. 10B is a graphic representation of the results of an MTT cytotoxicity assay that shows the viability of prohibitin siRNA transfected MDA cells treated with cisplatinum compared to mock transfected MDA controls.

FIG. 10C is a graphic representation of the results of an MTT cytotoxicity assay that shows the viability of prohibitin siRNA transfected MDA cells treated with taxol compared to mock transfected MDA controls.

FIG. 10D is a graphic representation of the results of an MTT cytotoxicity assay that shows the viability of prohibitin siRNA transfected MDA cells treated with etoposide compared to mock transfected MDA controls.

FIG. 10E is a graphic representation of the results of an MTT cytotoxicity assay that shows the viability of prohibitin siRNA transfected MDA cells treated with thiotepa compared to mock transfected MDA controls.

FIG. 10F is a graphic representation of the results of an MTT cytotoxicity assay that shows the viability of prohibitin siRNA transfected MDA cells treated with melphalan compared to mock transfected MDA controls.

FIG. 10G is a graphic representation of the results of an MTT cytotoxicity assay that shows the viability of prohibitin siRNA transfected MDA cells treated with mitoxantrone compared to mock transfected MDA controls.

FIG. 10H is a graphic representation of the results of an MTT cytotoxicity assay that shows the viability of prohibitin siRNA transfected MDA cells treated with vincristine compared to mock transfected MDA controls.

FIG. 11A is a graphic representation of the results of an MTT cytotoxicity assay that shows the viability of prohibitin siRNA transfected MDA cells treated with adriamycin compared to mock transfected MDA controls.

FIG. 11B is a graphic representation of the results of an MTT cytotoxicity assay that shows the viability of prohibitin siRNA transfected MDA cells treated with cisplatinum compared to mock transfected MDA controls.

FIG. 11C is a graphic representation of the results of an MTT cytotoxicity assay that shows the viability of prohibitin siRNA transfected MDA cells treated with taxol compared to mock transfected MDA controls.

FIG. 11D is a graphic representation of the results of an MTT cytotoxicity assay that shows the viability of prohibitin siRNA transfected MDA cells treated with vincristin compared to mock transfected MDA controls.

FIG. 12A is a graphic representation of the results of an MTT cytotoxicity assay that shows the viability of prohibitin siRNA transfected MDA cells treated with adriamycin compared to mock transfected MDA controls.

FIG. 12B is a graphic representation of the results of an MTT cytotoxicity assay that shows the viability of prohibitin siRNA transfected MDA cells treated with cisplatinum compared to mock transfected MDA controls.

FIG. 12C is a graphic representation of the results of an MTT cytotoxicity assay that shows the viability of prohibitin siRNA transfected MDA cells treated with taxol compared to mock transfected MDA controls.

FIG. 12D is a graphic representation of the results of an MTT cytotoxicity assay that shows the viability of prohibitin siRNA transfected MDA cells treated with thiotepa compared to mock transfected MDA controls.

FIG. 13A is a graphic representation of the results of an MTT cytotoxicity assay that shows the viability of prohibitin siRNA transfected MDA cells treated with adriamycin compared to mock transfected MDA controls.

FIG. 13B is a graphic representation of the results of an MTT cytotoxicity assay that shows the viability of prohibitin siRNA transfected MDA cells treated with cisplatinum compared to mock transfected MDA controls.

FIG. 13C is a graphic representation of the results of an MTT cytotoxicity assay that shows the viability of prohibitin siRNA transfected MDA cells treated with taxol compared to mock transfected MDA controls.

FIG. 13D is a graphic representation of the results of an MTT cytotoxicity assay that shows the viability of prohibitin siRNA transfected MDA cells treated with etoposide compared to mock transfected MDA controls.

FIG. 13E is a graphic representation of the results of an MTT cytotoxicity assay that shows the viability of prohibitin siRNA transfected MDA cells treated with mitoxantrone compared to mock transfected MDA controls.

FIG. 13F is a graphic representation of the results of an MTT cytotoxicity assay that shows the viability of prohibitin siRNA transfected MDA cells treated with vincristin compared to mock transfected MDA controls.

FIG. 13G is a graphic representation of the results of an MTT cytotoxicity assay that shows the viability of prohibitin siRNA transfected MDA cells treated with thiotepa compared to mock transfected MDA controls.

FIG. 13H is a graphic representation of the results of an MTT cytotoxicity assay that shows the viability of prohibitin siRNA transfected MDA cells treated with melphalan compared to mock transfected MDA controls.

FIG. 14A is a graphic representation of the results of an MTT cytotoxicity assay that shows the viability of prohibitin siRNA transfected MDA cells treated with adriamycin compared to mock transfected MDA controls.

FIG. 14B is a graphic representation of the results of an MTT cytotoxicity assay that shows the viability of prohibitin siRNA transfected MDA cells treated with cisplatinum compared to mock transfected MDA controls.

FIG. 14C is a graphic representation of the results of an MTT cytotoxicity assay that shows the viability of prohibitin siRNA transfected MDA cells treated with taxol compared to mock transfected MDA controls.

FIG. 14D is a graphic representation of the results of an MTT cytotoxicity assay that shows the viability of prohibitin siRNA transfected MDA cells treated with etoposide compared to mock transfected MDA controls.

FIG. 14E is a graphic representation of the results of an MTT cytotoxicity assay that shows the viability of prohibitin siRNA transfected MDA cells treated with mitoxantrone compared to mock transfected MDA controls.

FIG. 14F is a graphic representation of the results of an MTT cytotoxicity assay that shows the viability of prohibitin siRNA transfected MDA cells treated with vincristin compared to mock transfected MDA controls.

FIG. 14G is a graphic representation of the results of an MTT cytotoxicity assay that shows the viability of prohibitin siRNA transfected MDA cells treated with thiotepa compared to mock transfected MDA controls.

FIG. 14H is a graphic representation of the results of an MTT cytotoxicity assay that shows the viability of prohibitin siRNA transfected MDA cells treated with melphalan compared to mock transfected MDA controls.

FIG. 15A is a graphic representation of the results of an MTT cytotoxicity assay that shows the viability of prohibitin siRNA transfected MCF-7 cells treated with adriamycin compared to mock transfected MCF-7 controls.

FIG. 15B is a graphic representation of the results of an MTT cytotoxicity assay that shows the viability of prohibitin siRNA transfected MCF-7 cells treated with taxol compared to mock transfected MCF-7 controls.

FIG. 15C is a graphic representation of the results of an MTT cytotoxicity assay that shows the viability of prohibitin siRNA transfected MCF-7 cells treated with vincristin compared to mock transfected MCF-7 controls.

FIG. 15D is a graphic representation of the results of an MTT cytotoxicity assay that shows the viability of prohibitin siRNA transfected MCF-7 cells treated with cisplatinum compared to mock transfected MCF-7 controls.

FIG. 15E is a graphic representation of the results of an MTT cytotoxicity assay that shows the viability of prohibitin siRNA transfected MCF-7 cells treated with docetaxel compared to mock transfected MCF-7 controls.

FIG. 15F is a graphic representation of the results of an MTT cytotoxicity assay that shows the viability of prohibitin siRNA transfected MCF-7 cells treated with etoposide compared to mock transfected MCF-7 controls.

FIG. 15G is a graphic representation of the results of an MTT cytotoxicity assay that shows the viability of prohibitin siRNA transfected MCF-7 cells treated with mitoxantrone compared to mock transfected MCF-7 controls.

FIG. 15H is a graphic representation of the results of an MTT cytotoxicity assay that shows the viability of prohibitin siRNA transfected MCF-7 cells treated with melphalan compared to mock transfected MCF-7 controls.

FIG. 16A is a graphic representation of the results of an MTT cytotoxicity assay that shows the viability of prohibitin siRNA transfected SKOV3 cells treated with adriamycin compared to mock transfected SKOV3 controls.

FIG. 16B is a graphic representation of the results of an MTT cytotoxicity assay that shows the viability of prohibitin siRNA transfected SKOV3 cells treated with taxol compared to mock transfected SKOV3 controls.

FIG. 16C is a graphic representation of the results of an MTT cytotoxicity assay that shows the viability of prohibitin siRNA transfected SKOV3 cells treated with vincristin compared to mock transfected SKOV3 controls.

FIG. 16D is a graphic representation of the results of an MTT cytotoxicity assay that shows the viability of prohibitin siRNA transfected SKOV3 cells treated with cisplatinum compared to mock transfected SKOV3 controls.

FIG. 16E is a graphic representation of the results of an MTT cytotoxicity assay that shows the viability of prohibitin siRNA transfected SKOV3 cells treated with thiotepa compared to mock transfected SKOV3 controls.

FIG. 16F is a graphic representation of the results of an MTT cytotoxicity assay that shows the viability of prohibitin siRNA transfected SKOV3 cells treated with etoposide compared to mock transfected SKOV3 controls.

FIG. 16G is a graphic representation of the results of an MTT cytotoxicity assay that shows the viability of prohibitin siRNA transfected SKOV3 cells treated with mitoxantrone compared to mock transfected SKOV3 controls.

FIG. 16H is a graphic representation of the results of an MTT cytotoxicity assay that shows the viability of prohibitin siRNA transfected SKOV3 cells treated with melphalan compared to mock transfected SKOV3 controls.

FIG. 17A is a graphic representation of the results of an MTT cytotoxicity assay that shows the viability of prohibitin siRNA transfected SKOV3 cells treated with adriamycin compared to mock transfected SKOV3 controls.

FIG. 17B is a graphic representation of the results of an MTT cytotoxicity assay that shows the viability of prohibitin siRNA transfected SKOV3 cells treated with taxol compared to mock transfected SKOV3 controls.

FIG. 17C is a graphic representation of the results of an MTT cytotoxicity assay that shows the viability of prohibitin siRNA transfected SKOV3 cells treated with vincristin compared to mock transfected SKOV3 controls.

FIG. 17D is a graphic representation of the results of an MTT cytotoxicity assay that shows the viability of prohibitin siRNA transfected SKOV3 cells treated with cisplatinum compared to mock transfected SKOV3 controls.

FIG. 17E is a graphic representation of the results of an MTT cytotoxicity assay that shows the viability of prohibitin siRNA transfected SKOV3 cells treated with melphalan compared to mock transfected SKOV3 controls.

FIG. 17F is a graphic representation of the results of an MTT cytotoxicity assay that shows the viability of prohibitin siRNA transfected SKOV3 cells treated with mitoxantrone compared to mock transfected SKOV3 controls.

FIG. 17G is a graphic representation of the results of an MTT cytotoxicity assay that shows the viability of prohibitin siRNA transfected SKOV3 cells treated with thiotepa compared to mock transfected SKOV3 controls.

FIG. 17H is a graphic representation of the results of an MTT cytotoxicity assay that shows the viability of prohibitin siRNA transfected SKOV3 cells treated with etoposide compared to mock transfected SKOV3 controls.

FIG. 18A is a graphic representation of the results of an MTT cytotoxicity assay that shows the viability of prohibitin siRNA transfected SKOV3 cells treated with adriamycin compared to mock transfected SKOV3 controls.

FIG. 18B is a graphic representation of the results of an MTT cytotoxicity assay that shows the viability of prohibitin siRNA transfected SKOV3 cells treated with cisplatinum compared to mock transfected SKOV3 controls.

FIG. 18C is a graphic representation of the results of an MTT cytotoxicity assay that shows the viability of prohibitin siRNA transfected SKOV3 cells treated with taxol compared to mock transfected SKOV3 controls.

FIG. 18D is a graphic representation of the results of an MTT cytotoxicity assay that shows the viability of prohibitin siRNA transfected SKOV3 cells treated with vincristin compared to mock transfected SKOV3 controls.

FIG. 18E is a graphic representation of the results of an MTT cytotoxicity assay that shows the viability of prohibitin siRNA transfected SKOV3 cells treated with thiotepa compared to mock transfected SKOV3 controls.

FIG. 18F is a graphic representation of the results of an MTT cytotoxicity assay that shows the viability of prohibitin siRNA transfected SKOV3 cells treated with etoposide compared to mock transfected SKOV3 controls.

FIG. 18G is a graphic representation of the results of an MTT cytotoxicity assay that shows the viability of prohibitin siRNA transfected SKOV3 cells treated with mitoxantrone compared to mock transfected SKOV3 controls.

FIG. 18H is a graphic representation of the results of an MTT cytotoxicity assay that shows the viability of prohibitin siRNA transfected SKOV3 cells treated with melphalan compared to mock transfected SKOV3 controls.

### DETAILED DESCRIPTION OF THE INVENTION

The patent and scientific literature referred to herein establishes knowledge that is available to those of skill in the art. The issued U.S. patents, allowed applications, published foreign applications, and references, including GenBank database sequences, that are cited herein are hereby incorporated by reference to the same extent as if each was specifically and individually indicated to be incorporated by reference.

1.1 General

Aspects of the present invention provide methods and reagents for detecting and diagnosing chemotherapeutic drug-resistant cancer. Other aspects of the invention provide methods and reagents to treat and/or prevent cancer in a patient by increasing the sensitivity of the cancer cells to the chemotherapeutic drug(s). Additionally, aspects of the invention allow for the improved clinical identification and treatment of patients having chemotherapeutic drug-resistant neoplasms.

Accordingly, the present invention provides, in part, methods for diagnosing chemotherapeutic drug-resistance in a neoplastic cell. The method includes measuring a level of expression of prohibitin in a potentially chemotherapeutic drug resistant neoplastic cell sample, and comparing that level to the level of expression of prohibitin in a non-resistant control neoplastic cell of the same tissue type. The neoplastic cell samples are not cervical squamous cell carcinomas or are not derived therefrom. If the level of expression of prohibitin is greater in the neoplastic cell sample than in the non-resistant control neoplastic cell sample, chemotherapeutic drug-resistance is indicated. In some embodiments, the neoplastic cell sample and the non-resistant neoplastic cell are separated into fractions, and the cytoplasmic fractions are tested for prohibitin expression.

The invention also provides methods of treating and/or preventing cancer in a patient in need therefrom by increasing the sensitivity of the cancer cells to a chemotherapeutic treatment regime. The methods include administering an effective amount of a prohibitin-targeting agent to a cancer patient such that the prohibitin-targeting agent binds to prohibitin expressed by the neoplastic cells. Additionally, the patient is treated with a chemotherapeutic drug either simultaneously or subsequent to the administration of the prohibitin-targeting agent.

As used herein, a “neoplastic cell” is a cell that shows aberrant cell growth and/or contact inhibition, such as increased, uncontrolled cell proliferation. A neoplastic cell can be a hyperplastic cell, a cell from a cell line that shows a lack of contact inhibition when grown in vitro, a tumor cell when grown in vivo, or a cancer cell that is capable of metastasis in vivo. Alternatively, a neoplastic cell can be termed a “cancer cell.”

As used herein, “chemotherapeutic drug” means a pharmaceutical compound that kills a damaged cell such as a cancer cell. Cell death can be induced by the chemotherapeutic drug through a variety of means including, but not limited to, apoptosis, osmolysis, electrolyte efflux, electrolyte influx, cell membrane permeablization, and DNA fragmentation. Exemplary non-limiting chemotherapeutic drugs used for this purpose are adriamycin, cisplatinum, taxol, melphalan, daunorubicin, dactinomycin, bleomycin, fluorouracil, teniposide, vinblastin, vincristin, methotrexate, mitomycin, docetaxel, chlorambucil, carmustine, mitoxantrone, and paclitaxel.

The term “chemotherapeutic drug-resistance” as used herein encompasses the development of resistance or lack of response to a particular chemotherapeutic drug, class of chemotherapeutic drugs or multiple chemotherapeutic drugs by a cancer cell. Resistance can occur before or after treatment with a chemotherapy regime. The mechanism of development of chemotherapeutic drug resistance can occur by any means, such as pathogenic means, e.g., infection, particularly viral infection. Alternatively, chemotherapeutic drug resistance can be conferred by a mutation or mutations in one or several genes located either chromosomally or extrachromosomally. In addition, chemotherapeutic drug resistance can be conferred by selection of a certain phenotype by exposure to the chemotherapeutic drug or class of chemotherapeutic drugs, and then subsequent survival of the cell to the particular treatment. The terms, “chemotherapeutic drug-resistant” and “chemotherapeutic drug resistance,” are used to describe a neoplastic cell or a damaged cell that is chemotherapeutic drug-resistant due to either the classical mechanism (i.e., involving P-glycoprotein or another MDR protein) or an atypical (non-classical) mechanism that does not involve P-glycoprotein (e.g., one that involves the MRP1 chemotherapeutic drug resistance marker).

As used herein, the term “MDR protein” includes any of several integral transmembrane glycoproteins of the ABC type that are involved in multiple drug resistance. These include MDR 1 (P-glycoprotein or P-glycoprotein 1), an energy-dependent efflux pump responsible for decreased drug accumulation in chemotherapeutic drug-resistant cells. Examples of MDR 1 include human MDR 1 (see, e.g., database code MDR1_HUMAN, GenBank Accession No. P08183, 1280 amino acids (141.34 kD)). Other MDR proteins include MDR 3 (or P-glycoprotein 3), which is an energy-dependent efflux pump that causes decreased drug accumulation but is not capable of conferring drug resistance by itself. Non-limiting examples of MDR 3 include human MDR 3 (see, e.g., database code MDR3_HUMAN, GenBank Accession No. P21439, 1279 amino acids (140.52 kD). Other MDR-associated proteins participate in the active transport of drugs into subcellular organelles. Examples from human include MRP 1, Chemotherapeutic Drug Resistance-associated Protein 1, database code MRP_HUMAN, GenBank Accession No. P33527, 1531 amino acids (171.47 kD).

The present invention provides targeting agents that are used to detect the level of expression of prohibitin in a cell sample. As used herein, the term “targeting agent” means a molecule that can bind, associate, or hybridize with a target molecule in a specific manner. The mechanisms of binding to a target molecule include, e.g., hydrogen bonding, Van der Waals attractions, covalent bonding, ionic bonding, or hydrophobic interactions. In certain embodiments, a targeting agent is used to detect the level of expression of prohibitin in a neoplastic cell sample.

Aspects of the present invention also provide methods of detecting chemotherapeutic drug resistance in a patient. In some situations, a patient is identified when he/she no longer responds to the drug being used in his/her treatment. For example, a breast cancer patient in remission being treated with a chemotherapeutic agent (e.g., vincristin) may suddenly come out of remission, despite being treated with the chemotherapeutic agent. Unfortunately, such a patient is often found also to be unresponsive to other chemotherapeutic agents, including some to which the patient has never been exposed. Of course, after these patients become chemotherapeutic drug-resistant, their treatment to control their now-resurgent cancer or disease is difficult and may require more drastic therapies, such as radiotherapy, surgical resection, bone marrow transplantation, and/or amputation of necrotic tissue.

The method of the invention includes administering to a cancer patient a detectably labeled prohibitin-targeting agent and detecting the prohibitin-targeting agent that binds to expressed prohibitin using a detectable label linked to the prohibitin-targeting agent. As used herein, “detectably labeled” means that a targeting agent is operably linked to a moiety that is detectable. By “operably linked” is meant that the moiety is attached to the targeting agent by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

Some aspects of the present invention also allow an early diagnosis of chemotherapeutic drug resistance by detecting increased amounts of prohibitin in neoplastic cells of the patient. Such an early diagnosis allows patients who are initially drug responders and sensitive to drug treatment to be distinguished from those who are initially drug non-responders. Further, diagnostic procedures using prohibitin expression may also be used to follow the development and emergence of MDR neoplastic cells that are resistant to the treatment drug and that arise during the course of drug treatment, permitting health professionals to tailor their treatments accordingly.

The invention also provides methods of treating or preventing the growth of resistant or chemosensitive neoplasms in a patient in need thereof. The methods include administering to a patient an effective amount of prohibitin-targeting agent to the neoplasm or to a site in close proximity to the neoplasm. Additionally, the patient is administered a chemotherapeutic drug to kill the neoplastic cells after the cells have been targeted by the prohibitin-targeting agent to increase the chemosensitivity of the neoplastic cells to the chemotherapeutic drug. Alternatively, the targeting agent and the chemotherapeutic drug are administered simultaneously, e.g., each separately or as a single, linked therapeutic.

1.2 Cancer Cells for Diagnostic Methods

Cancer cells useful in the diagnostic methods of the invention or to be tested or treated by methods of the invention, can be obtained from any tissue including, but not limited to, breast, lung, bone, blood, skin, brain, gastrointestinal, lymphatic, hepatic, muscle, ovary, uterine, and kidney. Cancer cells can be obtained from tissues other than the tissue from which the cancer cell originally developed, as in the case of metastasized cancer cells. Moreover, cancer cells can be obtained from mammals including, but not limited to, human, non-human primates such as chimpanzee, mouse, rat, guinea pig, chinchilla, rabbit, pig, and sheep.

Alternatively, cancer cells can be obtained in the form of a cell line. The term “cell line,” as used herein, means any cell that has been isolated from the tissue of a host organism and propagated by artificial means outside of the host organism. Such cell lines can be chemotherapeutic drug-resistant or chemotherapeutic drug-sensitive. A cell line is isolated, or derived from, tissues such as prostatic tissue, bone tissue, blood, brain tissue, lung tissue, ovarian tissue, epithelial tissue, breast tissue, and muscle tissue. A cell line can be derived, produced, or isolated from a cancer cell type, e.g., melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, or thymoma. Cell lines can also be generated by techniques well known in the art (see, e.g., Griffin et. al., (1984) Nature 309(5963): 78-82). Useful, exemplary, and non-limiting cell lines include MCF7, MDA, SKOV3, OVCAR3, 2008, PC3, T84, HCT-116, H69, H460, HeLa, and MOLT4. Cell or cell lines not used in or treated by the methods of the invention are cervical squamous carcinomas, or are derived therefrom.

1.3 Targeting Agents

The present invention utilizes a prohibitin-targeting agent for use in diagnosing cancer and multi-drug resistant cancer in patients. Such agents are also used to increase the sensitivity of neoplasms to chemotherapeutic treatment. Additionally, the present invention utilizes prohibitin-targeting agents for use in preventing or treating chemotherapeutic drug-resistant neoplasms. As used herein, the term “prohibitin-targeting agent” refers to molecules that can specifically bind to prohibitin expressed in the cell. Prohibitin expression includes a nucleic acid expression such as messenger RNA (“mRNA”) that encodes prohibitin polypeptide or a fragment of the polypeptide. Prohibitin can be expressed as a polypeptide or as fragments of the polypeptide.

A prohibitin-targeting agent can be any particular molecule capable of binding to the prohibitin polypeptide, peptide fragments of prohibitin, prohibitin mRNA, or one or more prohibitin gene sequences. Non-limiting examples of targeting agents include antibodies, antibody prohibitin-binding fragments, nucleic acids, proteins, peptides, and peptidomimetic compounds. In some instances, targeting agents can be in the form of proteins (hereinafter termed “protein-targeting agents”). As used herein, the term “protein-targeting agents” means a protein molecule or polypeptide or peptide fragment thereof that can interact, bind, or associate with a molecule in a sample. Protein-targeting agents can also be nucleic acid aptamers that specifically bind to the prohibitin polypeptide, or a portion of the prohibitin polypeptide. Such protein-targeting agent is capable of binding a biological macromolecule such as a protein, nucleic acid, simple carbohydrate, complex carbohydrate, fatty acid, lipoprotein, and/or triacylglyceride. Exemplary protein targeting agents include natural ligands of a receptor, hormones, antibodies, and binding portions thereof. Techniques associated with the binding of ligands and hormones to proteins as targeting agents have been demonstrated previously (see, e.g., Cutting et al., (2004) J. Biomol. NMR. 30(2):205-10).

When the protein-targeting agents are antibodies or fragments of antibodies that specifically bind to prohibitin, they may be immobilized on a solid support such as an antibody array where the support can be a bead or flat surface similar to a slide. An antibody microarray can determine the MDR protein expression of a chemotherapeutic drug-resistant cancer cell sample and the MDR protein expression of a multi-drug-sensitive control cell of the same tissue type. Alternatively, antibodies can be free in solution. Antibodies can also be conjugated to a non-limiting material such as magnetic compounds, paramagnetic compounds, proteins, nucleic acids, antibody fragments, or combinations thereof. In some embodiments, antibodies are used to inhibit prohibitin to decrease the activity of the enzyme in a targeted cell, as an inhibitor, thereby increasing the chemosensitivity of the cell to chemotherapeutic treatments (see Lopez-Alemany et al. (2003) Am. J. Hematol. 72(4): 234-42).

Protein-targeting agents, including antibodies, can be detectably labeled. Useful labels include, without limitation, fluorophores (e.g., fluorescein (FITC), phycoerythrin, rhodamine), chemical dyes, or compounds that are radioactive, chemoluminescent, magnetic, paramagnetic, promagnetic, or enzymes that yield a product that may be colored, chemoluminescent, or magnetic. The signal is detectable by any suitable means, including spectroscopic, photochemical, biochemical, immunochemical, electrical, optical or chemical means. In certain cases, the signal is detectable by two or more means.

Labeled protein targeting agents allow detection of the level of expression of prohibitin in a cancer cell sample. For example, protein-targeting agents can be labeled for detection using chemiluminescent tags affixed to amino acid side chains. Useful tags include, but are not limited to, biotin, fluorescent dyes such as Cy5 and Cy3, and radiolabels (see, e.g., Barry and Soloviev (2000) Proteomics. 4(12): 3717-3726). Tags can be affixed to the amino terminal portion of a protein or the carboxyl terminal portion of a protein (see, e.g., Mattison and Kenney, (2002) J. Biol. Chem., 277(13): 11143-11148; Berne et al., (1990) J. Biol. Chem. 265(32): 19551-9). Indirect detection means can also be used to identify the cell markers. Exemplary but non-limiting means include detection of a primary antibody using a fluorescently labeled secondary antibody, or a secondary antibody tagged with biotin such that it can be detected with fluorescently labeled streptavidin.

The prohibitin-targeting agent may alternatively comprise a nucleic acid. As used herein, a “nucleic acid targeting agent” is defined as a nucleic acid capable of binding to a target nucleic acid of complementary sequence through one or more types of chemical bonds, usually through complementary base pairing, usually through hydrogen bond formation. Examples of a nucleic acid targeting agent include, but are not limited to, siRNA, antisense oligonucleotides, and ribozymes. “Nucleic acid” refers to a polymer comprising two or more nucleotides and includes single-, double-, and triple-stranded polymers. “Nucleotide” refers to both naturally occurring and non-naturally occurring compounds and comprises a heterocyclic base, a sugar, and a linking group, such as a phosphate ester. For example, structural groups may be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid may be substituted or modified, for example with methyl phosphonates or O-methyl phosphates. Bases and sugars can also be modified, as is known in the art. “Nucleic acid,” for the purposes of this disclosure, also includes “peptide nucleic acids” in which native or modified nucleic acid bases are attached to a polyamide backbone.

Moreover, a nucleic acid targeting agent may include natural (i.e., A, G, U, C, or T) or modified (7-deazaguanosine, inosine, etc.) bases. In addition, the bases in targeting agents may be joined by a linkage other than a phosphodiester bond, so long as it does not interfere with hybridization. Thus, nucleic acid targeting agents may be peptide nucleic acids in which the constituent bases are joined by peptide bonds rather than phosphodiester linkages. The nucleic acid targeting agents may be prepared by converting the RNA to cDNA using known methods (see, e.g., Ausubel et. al., Current Protocols in Molecular Biology, Wiley 1999). The targeting agents can also be cRNA (see, e.g., Park et. al., (2004) Biochem. Biophys. Res. Commun. 325(4): 1346-52).

Nucleic acid targeting agents can be produced from synthetic methods such as phosphoramidite methods, H-phosphonate methodology, and phosphite triester methods. Nucleic acid targeting agents can also be produced by PCR methods. Such methods produce cDNA and cRNA sequences complementary to the mRNA.

Nucleic acid targeting agents can be detectably labeled, with, e.g., fluorophores (e.g., fluorescein (FITC), phycoerythrin, rhodamine), chemical dyes, or compounds that are radioactive, chemoluminescent, magnetic, paramagnetic, promagnetic, or enzymes that yield a product that may be colored, chemiluminescent, or magnetic. The signal is detectable by any suitable means, including spectroscopic, photochemical, biochemical, immunochemical, electrical, optical or chemical means. In certain cases, the signal is detectable by two or more means. In certain embodiments, nucleic acid labels include fluorescent dyes, radiolabels, and chemiluminescent labels, which are examples that are not intended to limit the scope of the invention (see, e.g., Yu, et al., (1994) Nucleic Acids Res. 22(16): 3226-3232; Zhu, et al., (1994) Nucleic Acids Res. 22(16): 3418-3422).

Nucleic acid targeting agents can be detectably labeled using fluorescent labels. Non-limiting examples of fluorescent labels include 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein). These labels can be commercially obtained, e.g., from PerkinElmer Corp. (Boston, Mass.).

Other useful dyes are chemiluminescent dyes and can include, without limitation, biotin conjugated DNA nucleotides and biotin conjugated RNA nucleotides. Labeling of nucleic acid targeting agents can be accomplished by any means known in the art. (see, e.g., CyScribe™ First Strand cDNA Labeling Kit (#RPN6200, Amersham Biosciences, Piscataway, N.J.). The label can be added to the target nucleic acid(s) prior to, or after the hybridization. So called “direct labels” are detectable labels that are directly attached to, or incorporated into, the target nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore binds the biotin bearing hybrid duplexes providing a label that is easily detected. (see, e.g., Laboratory Techniques in Biochemistry and Molecular Biology, Vol. 24: Hybridization With Nucleic Acid Targeting agents, P. Tijssen, ed. Elsevier, N.Y., (1993)).

Nucleic acid targeting agents can also be immobilized on a solid support such as glass, polystyrene, nylon, and PVDF membrane. In these embodiments, the nucleic acid targeting agent is contacted by an isolated cell sample, and subsequently allowed to hybridize to the target nucleic acid in the sample. In certain embodiments, a microarray is utilized to detect prohibitin expression levels. Microarray technology has been utilized to determine the expression levels of various other genes, and the techniques are well known in the art (see, e.g., Zhang et al. (2004) Proc. Nat. Acad. Sci. USA. 101(39): 14168-14173).

Alternatively, expression levels for the prohibitin mRNA can be determined using other techniques known in the art, such as, but not limited to, quantitative RT-PCR and RNA blotting (see, e.g., Rehman et al. (2004) Hum. Pathol. 35(11): 1385-91; Yang et al. (2004) Mol. Biol. Rep. 31(4): 241-8). In these embodiments, the nucleic acid targeting agent comprises nucleic acid probes that are complementary to prohibitin mRNA sequences. The probes can be complementary to any region of the prohibitin mRNA provided that the probes allow for sufficient hybridization to the prohibitin mRNA. Such examples are not intended to limit the potential means for determining the expression of a gene marker in a breast cancer cell sample.

The prohibitin-targeting agent useful in the methods of the invention can be composed of multiple parts, herein termed “components.” The components can be conjugated to one another using techniques known in the art (see, e.g., U.S. Pat. Nos. 6,962,981, 4,935,233, 6,962,709, 6,958,361). For example, the prohibitin-targeting agent can have a cell-associating component. A useful cell-associating component is an antibody, or binding fragment thereof, such as Fv, F(ab′)₂, F(ab), Dab, and SC-Mab, that binds to cell surface expressed cancer cell markers such as Pgp-1, MRP1, BIP, BRCP, HSC70, nucleophosmin, vimentin, and HSP90. The cell-associating component can also be a compound that binds to a cell marker such as, but not limited to, an inhibitor of a cancer cell marker, peptide, peptidomimetic, ligand, or small molecule. As used herein, the term “inhibitor” means a molecule that prevents or interferes with a biomolecule, e.g., a protein, nucleic acid, or ribozyme, from completing or initiating a reaction. An inhibitor can inhibit a reaction by competitive, uncompetitive, or non-competitive means. Exemplary inhibitors include, but are not limited to, nucleic acids, proteins, peptides, peptidomimetic compounds, small molecules, chemicals, and analogs that mimic the binding site of an enzyme. As long as the interaction of the cell-associating component allows for cancer cell-specific targeting of the prohibitin-targeting agent, a compound is useful as a cell-associating component.

The prohibitin-targeting agent also can include a cell-internalization component that allows the prohibitin-targeting agent to enter into cell. For example, a cell-internalization component can be an agent such as a liposome or immunoliposome that allows for cell membrane fusion between the prohibitin-targeting agent and the cancer cell (see, e.g., Drummond, et al, (2005) Ann. Rev. Pharmacol. Toxicol. 45: 495-528).

The above-described components of the targeting agent can be conjugated together to form a single agent using conjugation techniques that are well known in the art (see, e.g., U.S. Pat. Nos. 4,833,077; 5,811,524; 5,958,765; 6,413,771; and 6,537,809). Conjugation of such components to other components or agents can be accomplished by, e.g., covalent bonding to non-limiting active groups such as carbonyls, carboxyls, amines, amides, hydroxyls, and sulfhydryls. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y. 1999).

The cell-internalization component can be a dendrimer conjugate, which is a spherical polymer (see, e.g., Tomalia, D. A., et al., (1990) Angew. Chem. Int. Ed. Engl. 29: 5305). Synthesis and utilization of dendrimers has been postulated in the art, and dendrimers have been utilized for chemotherapeutic drug targeting in vitro (see, e.g., P. Singh, et al., (1994) Clin. Chem. 40: 1845). The prohibitin-specific targeting component binds prohibitin or a portion thereof so as to decrease the activity of the prohibitin enzyme in the targeted cancer cell. The prohibitin-specific targeting component can be a nucleic acid that hybridizes specifically to sequences encoding prohibitin or a portion of the prohibitin polypeptide. Useful prohibitin-specific targeting components are peptides, peptidomimetic compounds, small molecules specifically designed to bind prohibitin, and inhibitors of prohibitin. The aforementioned compounds are not intended to limit the range of compounds that can serve as the prohibitin-specific targeting component, but are merely illustrative examples.

Alternatively, the prohibitin-targeting agent can be nucleic acid that specifically hybridizes to a segment or region of the prohibitin nucleic acids expressed in the cancer cells. Such nucleic acids typically are from 5 to 50 nucleotides in length. Useful nucleic acids are antisense oligonucleotides, ribozymes and siRNA. For example, ribonucleic acids used in RNAi to hybridize to target sequences can be of lengths between 10 to 20 bases, between 9 to 21 bases, between 7 to 23 bases, between 5 to 25 bases, between 25 to 35 bases, between 27 to 33 bases, and between 35 to 40 bases.

1.4 Aptamers as Prohibitin Targeting Agents

As described above, aptamers can be prohibitin-targeting agents. The term “aptamer,” used herein interchangeably with the term “nucleic acid ligand,” means a nucleic acid that, through its ability to adopt a specific three-dimensional conformation, binds to and has an antagonizing (i.e., inhibitory) effect on a target. The target according to the present invention is prohibitin, and hence the term prohibitin aptamer or nucleic acid ligand is used. Inhibition of the target by the aptamer may occur by binding of the target, by catalytically altering the target, by reacting with the target in a way which modifies/alters the target or the functional activity of the target, by covalently attaching to the target as in a suicide inhibitor, by facilitating the reaction between the target and another molecule.

Aptamers are comprised of multiple ribonucleotide units, deoxyribonucleotide units, or a mixture of both types of nucleotide residues. Aptamers may further comprise one or more modified bases, sugars or phosphate backbone units as described above.

Aptamers can be made by any known method of producing oligomers or oligonucleotides. For example, 2′-O-allyl modified oligomers that contain residual purine ribonucleotides, and bearing a suitable 3′-terminus such as an inverted thymidine residue (Ortigao et al., (1992) Antisense Res. Devel. 2:129-146) or two phosphorothioate linkages at the 3′-terminus to prevent eventual degradation by 3′-exonucleases, can be synthesized by solid phase beta-cyanoethyl phosphoramidite chemistry (Sinha et al., Nucleic Acids Res., 12:4539-4557 (1984)) on any commercially available DNA/RNA synthesizer. One method is the 2′-O-tert-butyldimethylsilyl (TBDMS) protection strategy for the ribonucleotides (Usman et al., (1987) J. Am. Chem. Soc., 109: 7845-7854), and all the required 3′-O-phosphoramidites are commercially available. In addition, aminomethylpolystyrene may be used as the support material due to its advantageous properties (McCollum and Andrus (1991) Tetrahedron Lett. 32:4069-4072). Fluorescein can be added to the 5′-end of a substrate RNA during the synthesis by using commercially available fluorescein phosphoramidites.

In general, an aptamer oligomer can be synthesized using a standard RNA cycle. Upon completion of the assembly, all base labile protecting groups are removed by an eight-hour treatment at 55° C. with concentrated aqueous ammonia/ethanol (3:1 v/v) in a sealed vial. The ethanol suppresses premature removal of the 2′-O-TBDMS groups that would otherwise lead to appreciable strand cleavage at the resulting ribonucleotide positions under the basic conditions of the deprotection (Usman et al., (1987) J. Am. Chem. Soc., 109: 7845-7854). After lyophilization, the TBDMS protected oligomer is treated with a mixture of triethylamine trihydrofluoride/triethylamine/N-methylpyrrolidinone for 2 hours at 60° C. to afford fast and efficient removal of the silyl protecting groups under neutral conditions (see Wincott et al., (1995) Nucleic Acids Res., 23:2677-2684). The fully deprotected oligomer can then be precipitated with butanol according to the procedure of Cathala et al. ((1990) Nucleic Acids Res., 18:201). Purification can be performed either by denaturing polyacrylamide gel electrophoresis or by a combination of ion exchange HPLC (Sproat et al., (1995) Nucleosides and Nucleotides, 14:255-273) and reversed phase HPLC. For use in cells, synthesized oligomers are converted to their sodium salts by precipitation with sodium perchlorate in acetone. Traces of residual salts may then be removed using small disposable gel filtration columns that are commercially available. As a final step the authenticity of the isolated oligomers may be checked by matrix assisted laser desorption mass spectrometry (Pieles et al., (1993) Nucleic Acids Res., 21:3191-3196) and by nucleoside base composition analysis.

Aptamers can also be produced through enzymatic methods, when the nucleotide subunits are available for enzymatic manipulation. For example, the RNA molecules can be made through in vitro RNA polymerase T7 reactions. They can also be made by strains of bacteria or cell lines expressing T7, and then subsequently isolated from these cells. As discussed below, the disclosed aptamers can also be expressed in cells directly using vectors and promoters.

An issue to be addressed in the diagnostic or therapeutic use of nucleic acids is the potential rapid degradation of oligonucleotides in their phosphodiester form in body fluids by intracellular and extracellular enzymes such as endonucleases and exonucleases before the desired effect is manifest. Certain chemical modifications of the nucleic acid ligand can be made to increase the in vivo stability of the nucleic acid ligand or to enhance or to mediate the delivery of the nucleic acid ligand (see, e.g., U.S. Pat. No. 5,660,985). For example, the stability of the aptamer can be greatly increased by the introduction of such modifications and as well as by modifications and substitutions along the phosphate backbone of the RNA. In addition, a variety of modifications can be made on the nucleobases themselves, which both inhibit degradation and which can increase desired nucleotide interactions or decrease undesired nucleotide interactions. Accordingly, once the sequence of an aptamer is known, modifications or substitutions can be made by the synthetic procedures described below or by procedures known to those of skill in the art.

Other modifications include the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). The aptamers may also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer may further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer may still further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil.

Examples of other modified base variants known in the art include, without limitation, e.g., 4-acetylcytidine, 5-(carboxyhydroxylmethyl) uridine, 2′-methoxycytidine, 5-carboxymethylaminomethyl-2-thioridine, 5-carboxymethylaminomethyluridine, dihydrouridine, 2′-O-methylpseudouridine, b-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N6-isopentenyladenosine, N—((9-b-D-ribofuranosyl-2-methylthiopurine-6-yl)carbamoyl)threonine, N—((9-b-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N—((9-b-D-ribofuranosylpurine-6-yl)carbamoyl)threonine, 2′-O-methyl-5-methyluridine, 2′-O-methyluridine, and wybutosine, 3-(3-amino-3-carboxypropyl)uridine.

Also included are the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941. Examples of modified nucleoside and nucleotide sugar backbone variants known in the art include, without limitation, those having, e.g., 2′ ribosyl substituents such as F, SH, SCH₃, OCN, Cl, Br, CN, CF₃, OCF₃, SOCH₃, SO₂, CH₃, ONO₂, NO₂, N₃, NH₂, OCH₂CH₂OCH₃, O(CH₂) 20N(CH₃)₂, OCH₂OCH₂N(CH₃)₂, O(C₁₋₁₀ alkyl), O(C₂₋₁₀ alkenyl), O(C₂₋₁₀ alkynyl), S(C₁₋₁₀ alkyl), S(C₂₋₁₀ alkenyl), S(C₂₋₁₀ alkynyl), NH(C₁₋₁₀ alkyl), NH(C₂₋₁₀ alkenyl), NH(C₂₋₁₀ alkynyl), and O-alkyl-O-alkyl. Desirable 2′ ribosyl substituents include 2′-methoxy(2′-OCH₃), 2′-aminopropoxy(2′ OCH₂CH₂CH₂NH₂), 2′-allyl (2′-CH₂—CH═CH₂), 2′-O-allyl (2′-O—CH₂—CH═CH₂), 2′-amino (2′-NH₂), and 2′-fluoro (2′-F). The 2′-substituent may be in the arabino (up) position or ribo (down) position.

Aptamers may be made up of nucleotides and/or nucleotide analogs such as described above, or a combination of both, or are oligonucleotide analogs. Aptamers may contain nucleotide analogs at positions, which do not affect the function of the oligomer to bind prohibitin.

There are several techniques that can be adapted for refinement or strengthening of the nucleic acid ligands binding to a particular target molecule or the selection of additional aptamers. One technique, generally referred to as “in vitro genetics” (see Szostak (1992) TIBS, 19:89), involves isolation of aptamer antagonists by selection from a pool of random sequences. The pool of nucleic acid molecules from which the disclosed aptamers may be isolated may include invariant sequences flanking a variable sequence of approximately twenty to forty nucleotides. This method has been termed Selective Evolution of Ligands by Exponential Enrichment (SELEX). Compositions and methods for generating aptamer antagonists of the invention by SELEX and related methods are known in the art and taught in, for example, U.S. Pat. Nos. 5,475,096 and 5,270,163. The SELEX process in general is further described in, e.g., U.S. Pat. Nos. 5,668,264, 5,696,249, 5,670,637, 5,674,685, 5,723,594, 5,756,291, 5,811,533, 5,817,785, 5,958,691, 6,011,020, 6,051,698, 6,147,204, 6,168,778, 6,207,816, 6,229,002, 6,426,335, and 6,582,918.

Other modifications useful for producing aptamers of the invention are known to one of ordinary skill in the art. Such modifications may be made post-SELEX process (modification of previously identified unmodified ligands) or by incorporation into the SELEX process. Finally, it has been observed that aptamers, or nucleic acid ligands, in general, are most stable, and therefore efficacious when 5′-capped and 3′-capped in a manner which decreases susceptibility to exonucleases and increases overall stability.

1.5 Methods of Targeting Prohibitin Targeting Agents

Prohibitin-targeting agents can be specifically targeted to a neoplasm to prevent detection of prohibitin activity in normal cells or in the serum of the patient. Likewise, prohibitin-targeting agents can be used to specifically decrease the level of expression of prohibitin in neoplastic cells. Targeting mechanisms include non-limiting techniques such as conjugating the prohibitin-targeting agent to an agent that binds preferentially to a cancer cell marker (hereinafter termed “cancer cell targeting components”). Cancer cell targeting components include, but are not limited to, antibodies or binding fragments thereof, nucleic acids, peptides, small molecules, and peptidomimetic compounds. Cancer cell targeting components can be conjugated directly to the prohibitin-targeting agent, for example, through covalent bonding to, e.g., carboxyl, phosphoryl, sulfhydryl, carbonyl, and hydroxyl groups using chemical techniques known in the art. Alternatively, cancer cell targeting components and prohibitin-targeting agents can be conjugated to functionalized chemical groups on non-limiting examples of inert supports such as polyethylene glycol, glass, synthetic polymers such as polyacrylamide, polystyrene, polypropylene, polyethylene, or natural polymers such as cellulose, Sepharose, or agarose, or conjugates with enzymes. Chemical conjugation techniques are well known in the art. Non-limiting examples of cancer cell markers that can be used for targeting of prohibitin-targeting agent include Pgp-1, MRP1, BIP, BRCP, HSC70, nucleophosmin, vimentin, and HSP90.

Alternatively, the prohibitin-targeting agent can be targeted to a neoplasm through variety of invasive procedures. In the context of some aspects of the present invention, such procedures include catheterization through an artery of a patient and depositing the prohibitin-targeting agent at the tumor site. A surgeon can also apply the prohibitin-targeting agent to the neoplasm by making a surgical incision into the patient at a site that allows access to the tumor for placement of the prohibitin-targeting agent into, onto, or in close proximity to, the tumor. In some instances, a subject can also be intubated with subsequent introduction of the prohibitin-targeting agent into the tumor site through the tube. In other embodiments, the prohibitin-targeting agent can be administered to a patient orally, subcutaneously, intramuscularly, intravenously, or interperitoneally.

The prohibitin-targeting agent can be targeted to a neoplasm by being incorporated into a liposome before it is administered or used. The term “liposome”, as used herein, refers to an artificial phospholipid bilayer vesicle. The liposome formulation can be used to facilitate lipid bilayer fusion with a target cell, thereby allowing the contents of the liposome or proteins associated with its surface to be brought into contact with the neoplastic cell. Liposomes can have antibodies associated with their bilayers that allow binding to targets on the neoplastic cell surface (hereinafter termed “immunoliposome”). Non-limiting examples of neoplastic cell targets to which such antibodies are specifically directed include Pgp-1, MRP1, BIP, BRCP, HSC70, nucleophosmin, vimentin, and HSP90. Antibodies for these cell markers can be obtained commercially (e.g., Research Diagnostics, Inc., Flanders, N.J.; and Abcam, Inc., Cambridge, Mass.).

1.6 Antibodies for Detection of Prohibitin

The invention also utilizes polyclonal and monoclonal antibodies for the detection of prohibitin. As used herein, the term “polyclonal antibodies” means a population of antibodies that can bind to multiple epitopes on an antigenic molecule. A polyclonal antibody is specific to a particular epitope on an antigen, while the entire pool of polyclonal antibodies can recognize different epitopes. In addition, polyclonal antibodies developed against the same antigen can recognize the same epitope on an antigen, but with varying degrees of specificity. Polyclonal antibodies can be isolated from multiple organisms including, but not limited to, rabbit, goat, horse, mouse, rat, and primates. Polyclonal antibodies can also be purified from crude serums using techniques known in the art (see, e.g., Ausubel, et al., Current Protocols in Molecular Biology Vol. 1, pp. 4.2.1-4.2.9, John Wiley & Sons, Inc., 1996).

The term “monoclonal antibody”, as used herein, refers to an antibody obtained from a population of substantially homogenous antibodies, i.e., the individual antibodies comprising the population are identical except for possible naturally occurring mutations that may be present in minor amounts. Monoclonal antibodies are highly specific, being directed against a single antigenic site. By their nature, monoclonal antibody preparations are directed to a single specific determinant on the target. Novel monoclonal antibodies or fragments thereof mean in principle all immunoglobulin classes such as IgM, IgG, IgD, IgE, IgA, or their subclasses or mixtures thereof. Non-limiting examples of subclasses include the IgG subclasses IgG1, IgG2, IgG3, IgG2a, IgG2b, IgG3, or IgGM. The IgG subtypes IgG1/κ and IgG2b/κ are also included within the scope of the present invention.

The monoclonal antibodies herein include hybrid and recombinant antibodies produced by splicing a variable (including hypervariable) domain of an anti-prohibitin antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567; Mage and Lamoyi, in Monoclonal Antibody Production Techniques and Applications, (Marcel Dekker, Inc., New York 1987, pp. 79-97). Thus, the modified “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method (see, e.g., Kohler and Milstein (1975) Nature 256:495) or can be made by recombinant DNA methods (U.S. Pat. No. 4,816,567). The monoclonal antibodies can also be isolated from phage libraries generated using the techniques described in the art (see, e.g., McCafferty et al. (1990) Nature 348:552-554).

Alternative methods for producing antibodies can be used to obtain high affinity antibodies. Antibodies for prohibitin can be obtained from human sources such as serum. Additionally, monoclonal antibodies can be obtained from mouse-human heteromyeloma cell lines by techniques known in the art (see, e.g., Kozbor (1984) J. Immunol. 133, 3001; Boerner et al., (1991) J. Immunol. 147:86-95). Methods for the generation of human monoclonal antibodies using phage display, transgenic mouse technologies, and in vitro display technologies are known in the art and have been described previously (see, e.g., Osbourn et al. (2003) Drug Discov. Today 8: 845-51; Maynard and Georgiou (2000) Ann. Rev. Biomed. Eng. 2: 339-76; U.S. Pat. Nos. 4,833,077; 5,811,524; 5,958,765; 6,413,771; and 6,537,809).

Antibodies are used to bind to prohibitin to decrease the activity of prohibitin in cancer cells. In some aspects of the invention, the antibody binds to prohibitin in domains vital to its activity. For instance, monoclonal or polyclonal antibodies directed to its calcium-binding domain can decrease the activity of prohibitin sufficiently to produce a desired inhibitory effect. Also, antibodies can be used to decrease the interaction of prohibitin with various proteins in the endoplasmic reticulum or Golgi complex. Techniques for inhibiting protein activity using antibodies are generally known in the art, and have been utilized to inhibit proteins such as PLTP, CETP, and other cell surface and intracellular proteins (see, e.g., Saito et al. (1999) J. Lipid Res. 40: 2013-2021; Cui et al. (2003) Eur. J. Biochem. 270: 3368-3376; Siggins et al. (2003) J. Lipid Res. 44: 1698-1704; Du et al. (1996) J. Biol. Chem. 271(13): 7362-7367).

1.7 RNA Interference

Aspects of the invention further enables the treatment of a neoplasm within a patient, such neoplasms may be chemotherapeutic drug-resistant neoplasms, which are treated by increasing the sensitivity of the neoplasm to a chemotherapeutic drug using, e.g., RNA interference (“RNAi”). As used herein, the term “RNA interference” refers to the blocking or preventing of cellular production of a particular protein by stopping the mechanisms of translation using small RNAs that hybridize to complementary sequences in a target mRNA. RNAi is essentially a type of antisense strategy for preventing RNA translation, even though the technology has slightly different mechanisms of action than general antisense strategies. Antisense RNA strategies utilize the single-stranded nature of mRNA in a cell to block or interfere with translation of the mRNA into a protein. Antisense technology has been the most commonly described approach in protocols to achieve gene-specific interference. For antisense strategies, stoichiometric amounts of single-stranded nucleic acid complementary to the messenger RNA for the gene of interest are introduced into the cell.

The RNA may comprise one or more strands of polymerized ribonucleotide. It may include modifications as described supra.

Methods of using RNA to inhibit gene expression are well known in the art (see e.g., U.S. Pat. No. 6,506,559). Typically, complementary RNA sequences that can hybridize to a specific region of the target RNA are introduced into the cell. RNA annealing to the target transcripts allows the internal machinery of the cell to cut the dsRNA sequences into short segments. It is this machinery that allows sub-stoichiometric numbers of siRNA molecules to be used to silence a particular gene. Such mechanisms have been utilized in in vitro and in vivo studies of human genes (see, e.g., Mizutani et al. (2002) J. Biol. Chem. 277(18): 15859-64; Wang et al. (2005) Breast Cancer Res. 7(2): R220-8). In particular, the c-myc gene was inhibited in MCF7 breast cancer cell lines using the RNA interference technique (see Wang et al. (2005) Breast Cancer Res. 7(2): R220-8).

Interfering RNAs can be prepared by any means known in the art. For example, they can be synthetically produced using the Expedite™ Nucleic Acid Synthesizer (Applied Biosystems, Foster City, Calif.) or other similar devices (see, e.g., Applied Biosystems, Foster City, Calif.). Synthetic oligonucleotides also can be produced using methods well known in the art such as phosphoramidite methods (see, e.g., Pan et. al., (2004) Biol. Proc. Online. 6:257-262), H-phosphonate methodology (see, e.g., Agrawal et. al., (1987) Tetrahedron Lett. 28(31): 3539-3542) and phosphite triester methods (Finnan et al. (1980) Nucleic Acids Symp. Ser. (7): 133-45).

1.8 Liposome-Mediated Delivery

Another strategy that may be employed for delivery of prohibitin-targeting agent is the use of liposomes which have also been used for the targeted delivery of chemotherapeutic drugs, toxins, and labels (see, e.g., Pakunlu et al. (2004) Cancer Res. 64(17): 6214-24; Shimizu et al. (2002) Biol. Pharm. Bull. 25(6): 783-6; Zheng and Tan (2004) World J. Gastroenterol. 10(17): 2563-6). Liposome formulations for the delivery of chemotherapeutics and siRNA can be obtained from commercial suppliers, e.g., Eurogentec, Ltd. (Southampton, Hampshire, UK). In addition, methods for producing liposome micelle/chemotherapeutic formulations are well known in the art. For example, therapeutic drug micelles can be formed by combining a therapeutic drug and a phosphatidyl glycerol lipid derivative (PGL derivative). Briefly, the therapeutic drug and PGL derivative are mixed in a range of 1:1 to 1:2.1 to form a therapeutic drug mixture. Alternatively, the range of therapeutic drug to PGL derivative is 1:1.2; or 1:1.4; or 1:1.5; or 1:1.6; or 1:1.8 or 1:1.9 or 1:2.0 or 1:2.1. The mixture is then combined with an effective amount of at least a 20% organic solvent such as an ethanol solution to form micelles containing the therapeutic drug.

Prohibitin targeting agents can be incorporated into the membrane of the liposome by any mechanisms known in the art (see, e.g., Pakunlu et al. (2004) Cancer Res. 64(17): 6214-24; Shimizu et al. (2002) Biol. Pharm. Bull. 25(6): 783-6; Zheng and Tan (2004) World J. Gastroenterol. 10(17): 2563-6). In addition, prohibitin-targeting agents can be associated with the outside of a liposome through covalent linkages to PEG polymers (see, e.g., Medina et al. (2004) Curr. Pharm. Des. 10(24): 2981-9). Furthermore, targeting agents can be incorporated into the hydrated inner compartment of the liposome (see, e.g., Medina et al. (2004) Curr. Pharm. Des. 10(24): 2981-9). A combination of the above mentioned liposome delivery methods can be used in a therapeutic composition.

Alternatively, modified LDL may be used as tumor-specific ligands in targeting liposomal formulations containing prohibitin-targeting agents. For example, folate-coupled liposomes can be used to target therapeutics to tumors, which overexpress the folate receptor (Lee and Low (1994) J. Biol. Chem. 269: 3198-204; Lee and Low (1995) Biochim. Biophys. Acta 1233: 134-44; Rui et al. (1998) J. Am. Chem. Soc. 120: 11213-18; Gabizon et al. (1999) Bioconj. Chem. 10: 289-98). Transferrin has been employed as a targeting ligand to direct liposomal drugs to various types of cancer cell in vivo (Ishida and Maruyama (1998) Nippon Rinsho 56: 657-62; Kirpotin et al. (1997) Biochem. 36: 66-75).

Immunoliposomes are also useful for delivery. Immunoliposomes incorporate antibodies against tumor-associated antigens into their membranes, which carry the therapeutic agent, such as the prohibitin-targeting agent, or an enzyme that activates an otherwise inactive prodrug (see, e.g., Lasic et al. (1995) Science 267: 1275-76). Immunoliposomal drugs have been used to successfully target and enhance anti-cancer efficacy (see, e.g., Maruyama et al. (1990) J. Pharm. Sci. 74: 978-84); Maruyama et al. (1995) Biochem. Biophys. Acta 1234: 74-80; Otsubo et al. (1998) Antimicrob. Agents Chemother. 42: 40-44; Lopes de Menezes et al. (1998) Cancer Res. 58: 3320-30).

Methods for inclusion of an antibody or tumor targeting ligand into the micelle formulation to produce immunoliposomes are known in the art and described further below. For example, methods for preparation and use of immunoliposomes are described in U.S. Pat. Nos. 4,957,735, 5,248,590, 5,464,630, 5,527,528, 5,620,689, 5,618,916, 5,977,861, 6,004,534, 6,027,726, 6,056,973, 6,060,082, 6,316,024, 6,379,699, 6,387,397, 6,511,676 and 6,593,308.

PEG-immunoliposomes with anti-transferring antibodies coupled to the distal ends of the PEG preferentially associate with C6 glioma cells in vitro and significantly increased gliomal doxorubicin uptake after treatment with the tumor-specific long-circulating liposomes containing doxorubicin (Eavarone et al. (2000) J. Biomed. Mater. Res. 51: 10-14).

1.9 Diagnostic Methods for Detection of Prohibitin

Aspects of the invention allow for the identification of chemotherapeutic resistance or MDR cancers and patients having MDR neoplastic cells. For example, where the patient identified as having such cells is a patient in remission of cancer or is being treated for cancer (e.g., a patient suffering from breast cancer, ovarian cancer, prostate cancer, leukemia, etc.), the invention allows identification of these patients prior to resurgence and/or progression of their cancer, as well as enables the monitoring of these patients during treatment with a drug, such that the treatment regimen can be altered.

The diagnostic applications of the invention include probes and other detectable agents that are described above. In certain embodiments, a cell sample is isolated from a patient, for example, by way of a biopsy of the potentially MDR cancerous tissue. Cell samples can also be isolated by non-limiting means such as surgical resection and tissue aspiration. In addition, the cell sample can be isolated from biological fluids including, but not limited to, blood, bile, urine, lacremal secretions, serum, and lymph. The methods of diagnosis also utilize a normal control cell sample to provide a level of expression that normally exists in a cell sample. The normal control cell sample can be obtained from sources including, but not limited to, tissue banks containing frozen tissues from normal subjects, cadavers, healthy subjects, and cell lines.

A level of expression of prohibitin is then measured in a potentially neoplastic cell sample and in a normal control cell sample of the same tissue as the cell sample. The level of expression for prohibitin can be determined using prohibitin-targeting agents that bind to the prohibitin protein or to prohibitin-encoding nucleic acid sequences, as described above. The prohibitin-targeting agents can be detectably labeled as described above. They can be bound by an “indirect label”, which is attached to another molecule that recognizes the prohibitin/prohibitin-targeting agent complex. Furthermore, the proteins from the cell sample can be labeled by methods known in the art, and then the labeled prohibitin is bound by the prohibitin-targeting agent.

In some embodiments, the method of diagnosing MDR in a cell sample includes comparing the levels of expression of prohibitin in a potentially MDR cell sample to the level of expression of prohibitin in a normal control cell sample. Comparisons can be made using techniques known in the art. Statistical methods of determining differences in protein expression include, but are not limited to, the Student T test. Statistically significant increases in the levels of expression of prohibitin in a potentially neoplastic cell sample indicate the presence of an MDR neoplastic cell in the cell sample.

2.0 Pharmaceutical Formulations and Therapeutic Methods

The present invention provides for both prophylactic and therapeutic methods of treating a subject having a neoplasm by increasing the sensitivity of the neoplasm to the chemotherapeutic treatment chosen by the physician. In certain cases, the present invention provides methods, both therapeutic and prophylactic, of treating a subject that suffers from a chemotherapeutic drug resistant neoplasm. For both non-resistant cancer and resistant cancer, administration of a prophylactic agent can occur prior to the manifestation of symptoms characteristic of the neoplasm, such that development of the neoplasm is prevented or, alternatively, delayed in its progression. In general, the prophylactic or therapeutic methods comprise administering to the patient an effective amount of a compound which comprises a prohibitin binding component that is capable of binding to prohibitin present in neoplastic, and particularly chemotherapeutic drug-resistant neoplastic, cells and which compound is linked to a therapeutic component. The prohibitin binding component or agent binds to the prohibitin expressed in or on the neoplastic cells and inhibitor prevents prohibitin activity in the cells, thereby rendering the cells susceptible to a chemotherapeutic treatment.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. (2005). For systemic administration, intramuscular, intravenous, intraperitoneal, and subcutaneous injection can be performed. For injection, the compounds of the invention are formulated in liquid solutions in physiologically compatible buffers, such as Hank's solution or Ringer's solution. In addition, the compounds may be formulated in solid or lyophilized form and dissolved or suspended immediately prior to use.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

For oral administration, the pharmaceutical compositions may take the form of, for example, tablets or capsules prepared by conventional means with pharmaceutically acceptable excipients such as targeting agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prohibitin-binding agents and/or chemotherapeutic drugs may also be formulated into rectal compositions such as suppositories or retention enemas, e.g., containing conventional suppository bases such as cocoa butter or other glycerides.

In addition to the therapeutic formulations described previously, the prohibitin-binding agents and/or chemotherapeutic drugs may also be formulated as a depot preparation. Such long acting formulations may be administered by implantation (for example, subcutaneously or intramuscularly) or by intramuscular injection. Thus, for example, the therapeutic formulations of the invention may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology utilizes microspheres of precapillary size which can be injected via a coronary catheter into any selected part of the e.g. heart or other organs without causing inflammation or ischemia. The administered therapeutic formulation is slowly released from these microspheres and taken up by surrounding tissue cells (e.g. endothelial cells).

The therapeutic compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

2.1 Prohibitin-Directed Cancer Therapies

The invention provides treatments that increase the sensitivity of cancer cells to chemotherapeutic drugs. Moreover, the invention provides for treatment or prevention of multi-drug-resistant cancer, including, but not limited to, neoplasms, tumors, or metastases, and particularly chemotherapeutic drug-resistant forms thereof by the administration of therapeutically or prophylactically effective amounts of anti-prohibitin antibodies or nucleic acid molecules encoding the antibodies. In addition, prohibitin therapies include nucleic acids complementary to a sequence encoding the prohibitin protein. Prohibitin therapies are utilized to decrease the activity of prohibitin in a cancer cell, thereby improving the efficacy of the treatment regime, and, in some instances, changing the chemotherapeutic drug-resistant phenotype of the cancer.

Examples of types of cancer and proliferative disorders that can be treated with the prohibitin-targeted therapeutic formulations of the invention include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma (but not including squamous cell carcinomas of the cervix or of cervical origin), basal cell carcinoma, adenocarcinoma, renal cell carcinoma, hepatoma, Wilms' tumor, cervical cancer (excluding cervical squamous cell carcinoma), uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. Therapeutic formulations of the invention can be administered to individuals with breast cancer (e.g., breast adenocarcinoma, breast carcinoma, ductal carcinoma in situ, ductal carcinoma, invasive ductal carcinoma, Paget's Disease of the Nipple, lobular carcinoma, lobular carcinoma in situ, invasive lobular carcinoma, inflammatory breast cancer, medullary carcinoma, tubular carcinoma, cribriform carcinoma, papillary carcinoma, phyllodes tumor). Additionally, therapeutic formulations of the invention can be administered to a subject suffering from ovarian cancer (e.g., serous carcinoma, ovarian adenocarcinoma, mucinous carcinoma, endometrioid carcinoma, clear cell carcinoma, Brenner carcinoma, mature cystic teratoma, monodermal teratoma, immature teratoma, dysgerminoma, embryonal carcinoma, granulosa cell carcinoma). The treatment and/or prevention of cancer or of chemotherapeutic drug-resistant cancer includes, but is not limited to, alleviating symptoms associated with cancer, the inhibition of the progression of cancer, the promotion of the regression of cancer, and the promotion of the immune response.

The prohibitin therapeutic formulations according to the invention can be administered in combination with other types of cancer treatments (e.g., radiation therapy, chemotherapy, hormonal therapy, immunotherapy and anti-tumor agents). Examples of anti-tumor agents include, but are not limited to, ifosfamide, paclitaxel, taxanes, topoisomerase I inhibitors (e.g., CPf-11, topotecan, 9-AC, and GG-211), gemcitabine, vinorelbine, oxaliplatin, 5-fluorouracil (5-FU), leucovorin, vinorelbine, Actinomycin, Adriamycin, Altretamine, Asparaginase, Bleomycin, Busulfan, Capecitabine, Carboplatin, Carmustine, Cisplatin, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristin, Vinorelbine., and temodal. These drugs are commercially obtainable, e.g., from ScienceLab.com, Inc. (Kingwood, Tex.). Physician administered treatment with these chemotherapeutic drugs is well known in the art (see, e.g., Capers et al., (1993) Hosp. Pharm. 28(3):206-10). Prohibitin-targeting agents can be administered to a patient for the prevention or treatment of chemotherapeutic drug resistance prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of the anti-tumor agent to the subject.

Prohibitin-targeted therapeutic formulations described herein, may be administered to a subject for the prevention or treatment of chemotherapeutic drug resistance prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of chemotherapeutic drugs described herein. Nucleic acids complementary to prohibitin messenger RNA are administered to a subject prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of chemotherapeutic drugs.

2.2 Kits for Detecting Chemotherapeutic Drug Resistance

Aspects of the invention additionally provide kits for detecting chemotherapeutic drug resistance in neoplastic cells. The kits include probes for the detection of prohibitin and probes for the detection of MRP 1, BRCP, p53, vimentin, α-enolase, nucleophosmin, and HSC70. During the course of chemotherapeutic treatment, monitoring of prohibitin, and other MDR-associated markers described herein, provides valuable information regarding the efficacy of the treatment and regarding the avoidance of the development of chemotherapeutic drug resistance.

The kit comprises a labeled compound or agent capable of detecting prohibitin protein in a biological sample, as well as means for determining the amount of prohibitin in the sample, and means for comparing the amount of prohibitin in the sample with a standard (e.g., non-MDR neoplastic cells or normal non-neoplastic cells) from the same tissue as the biological sample. The compound or agent can be packaged in a suitable container. The kit can further comprise instructions for using the compounds or agents to detect prohibitin protein, as well as other MDR-associated markers. Such a kit can comprise, e.g., one or more antibodies that bind specifically to at least a portion of a prohibitin protein on a neoplastic cell.

For example, the kit can contain nucleic acids that are capable of detecting prohibitin expression in a cell sample, as described above. Non-limiting examples of nucleic acids include single-stranded RNA, double-stranded RNA, double-stranded DNA, single-stranded DNA, and RNA-DNA hybrids. Such nucleic acids can include ribozymes, oligonucleotides, RNAi, antisense RNA, and combinations thereof. Furthermore, nucleic acids can be labeled as described supra.

The kit also contains a probe for detection of MDR protein expression, which indicates the presence of chemotherapeutic drug resistance. These probes advantageously allow health care professionals to obtain an additional data point to determine whether chemotherapeutic drug resistance exists. The probes can be labeled proteins, antibodies, or fragments thereof, or aptamers, capable of binding at least a portion of the chemotherapeutic drug resistance markers. Additionally, the probes can be nucleic acids capable of hybridizing to a region of an MDR or chemotherapeutic drug resistance marker such as MRP 1, BRCP, p53, vimentin, α-enolase, nucleophosmin, or HSC70. Any known MDR proteins known in the art can be used in the present aspect of the invention (see, e.g., Ojima et al. (2005) J. Med. Chem. 48(6): 2218-28; Matsumoto et al. (2005) J. Med. Invest. 52(1-2):41-8).

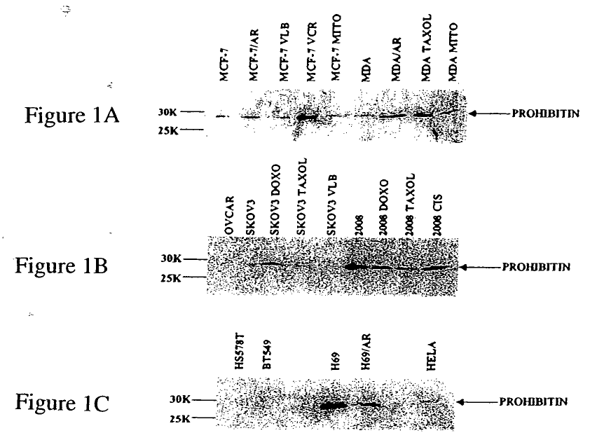

To demonstrate the methods according to the invention, a prohibitin-targeting agent was prepared and tested for its ability to increase the sensitivity of various cancer cell samples to chemotherapeutic drugs. As a first step to elucidating the role that prohibitin has in chemotherapeutic drug resistance, the levels of expression of prohibitin were determined in resistant and non-resistant MCF-7 and MDA breast cancer cell lines. Cell extracts were prepared from resistant and nonresistant cell lines, and immunoblotted using anti-prohibitin antibodies according to procedures described below. The non-resistant and resistant MCF-7 and MDA cells showed expression of a protein at approximately 28 kD, which corresponds to previous reports concerning prohibitin protein expression (FIG. 1A; see Dell'Orco et al. (1996) Exp. Gerontol. 31(1-2): 245-52). Of particular interest, MCF-7 cell lines resistant to vincristin and adriamycin had higher prohibitin expression levels than non-resistant MCF-7 cell lines (FIG. 1A). The increased levels of expression for prohibitin in MCF-7 resistant cell lines suggested that prohibitin could be a cell marker for chemotherapeutic drug resistance in breast cancer cells lines.

In addition to MCF-7 cell lines, the breast adenocarcinoma cell line MDA showed differential prohibitin expression that was dependent on whether the cells had developed resistance to a particular chemotherapeutic drug. MDA cell lines resistant to adriamycin, taxol, and mitoxantrone had significantly increased levels of expression of prohibitin protein as compared to non-resistant MDA cell lines (FIG. 1A). These results confirmed the results of the MCF-7 experiments, indicating that prohibitin is a marker for chemotherapeutic drug resistance in certain cancer types.

Additionally, ovarian cancer cell lines were tested to determine the effects of chemotherapeutic drug resistance on prohibitin expression. In the case of SKOV3 cell lines, chemotherapeutic resistance to doxorubicin (DOXO) was accompanied by increased expression of prohibitin (FIG. 1B). Other chemotherapeutic drugs showed less obvious changes in expression. 2008 cells showed variable prohibitin expression (FIG. 1B). The levels of expression of protein in various other cell lines is shown in FIG. 1C.

To determine the potential for utilizing prohibitin silencing in treating or improving the efficacy of certain chemotherapeutic treatments, short nucleotide sequences were designed using standard methodologies known in the art (see, e.g., RNAi Designer Resources, Invitrogen Corp., Carlsbad, Calif.). One sequence was designed that corresponded to a region that is highly specific for the prohibitin mRNA (Table 1). An additional sequence was designed that corresponded to the sense strand of the prohibitin gene (Invitrogen Corp., Carlsbad, Calif.). All sequences are shown in Table 1.

TABLE 1

Small Interfering RNA Duplexes
Targeting Prohibitin

| | | SEQ. ID |
|---|---|---|
| siRNA Duplex | Sequence | NO: |
| Prohibitin siRNA | 5′-UAACAGACAGACCACUUCC-3′ | 1 |
| (Antisense) | | |
| Prohibitin siRNA | 5′-GGAAGUGGUCUGUCUGUUA-3′ | 2 |
| (Sense) | | |

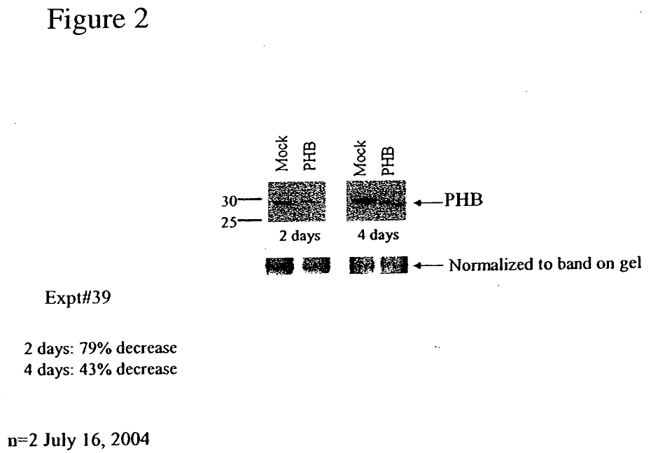

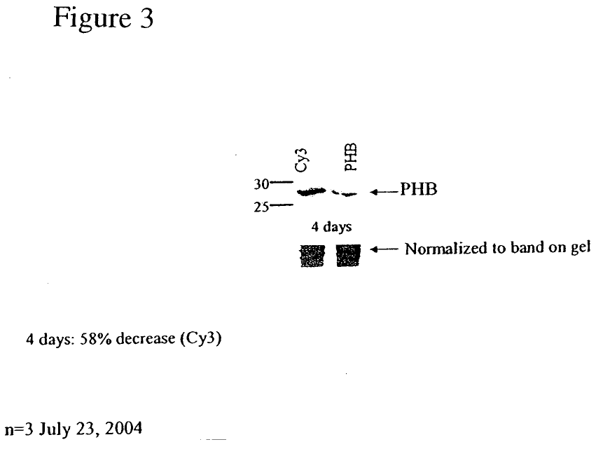

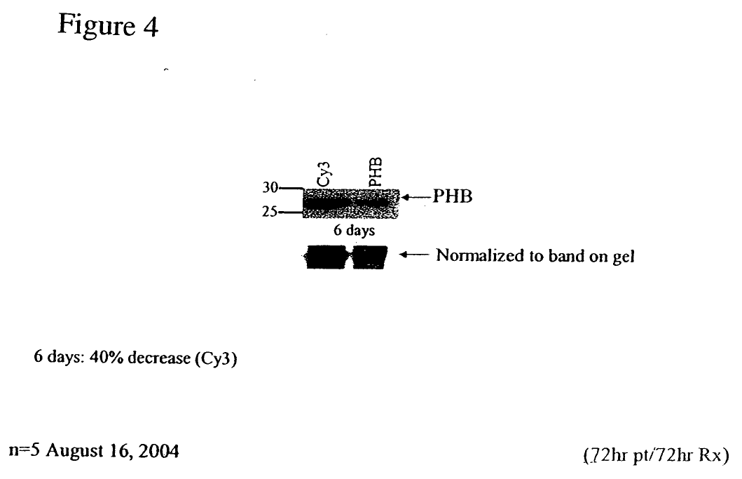

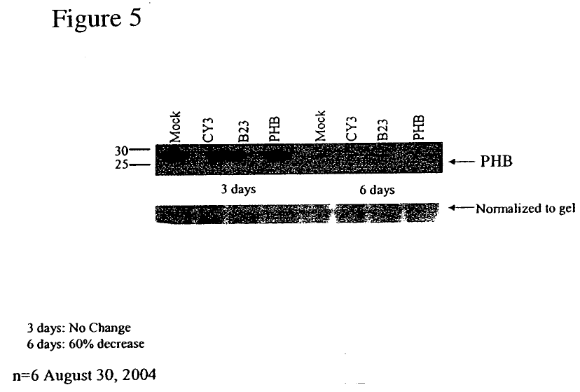

The prohibitin siRNA sequences were used in silencing experiments. Prohibitin expression was decreased after treatment with prohibitin siRNA in MDA cell lines by 79% two days post-treatment and 43% four days post-treatment (FIG. 2). These results were confirmed by additional siRNA studies on MDA cell lines in which prohibitin expression was still decreased by 58% on the fourth day after prohibitin siRNA treatment (FIG. 3). MDA cell lines were also treated with Cy3 siRNA as a control. Cy3 control siRNA affected prohibitin expression by 40% (FIG. 4). However, prohibitin siRNA had an even greater effect on prohibitin expression. FIG. 5 shows the effects of siRNA experiments utilizing sequences targeting B23, Cy3, and prohibitin. When compared to mock silencing, prohibitin siRNA decreased prohibitin expression by an additional 60%. Nucleophosmin and Cy3 also decreased prohibitin expression as well.

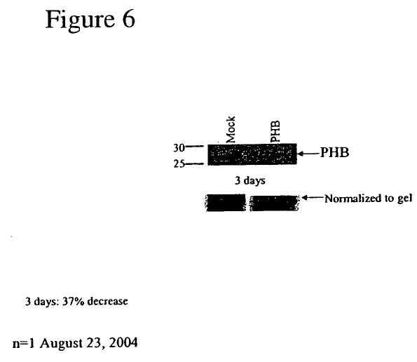

The results obtained in MDA cell lines were confirmed in MCF-7 cell lines. The MCF-7 and MDA cell lines were derived from breast adenocarcinomas. The MCF-7 cell line treated with prohibitin specific siRNA showed a 37% decrease in prohibitin expression as compared to mock siRNA treated cell lines (FIG. 6).

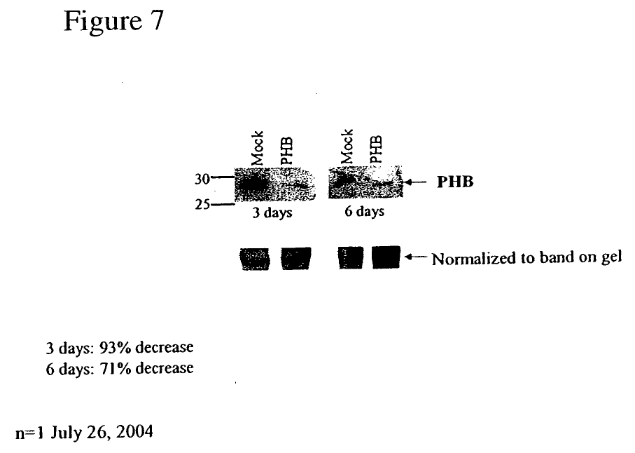

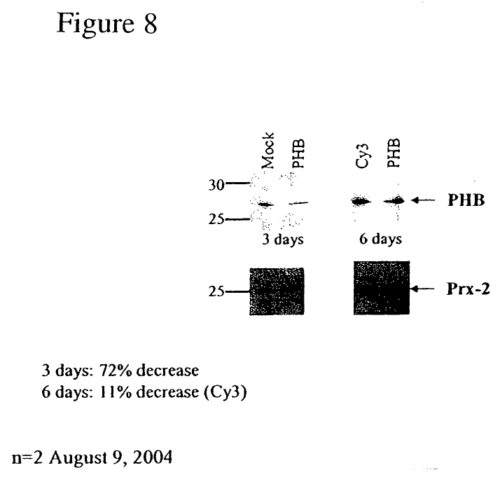

Ovarian cancer cell lines were also treated with mock siRNA and prohibitin-specific siRNA. SKOV3 ovarian cell lines showed nearly 93% decrease in prohibitin three days after treatment, and a 71% decrease in prohibitin six days after treatment (FIG. 7). These results were confirmed, albeit with increased prohibitin expression (FIG. 8).

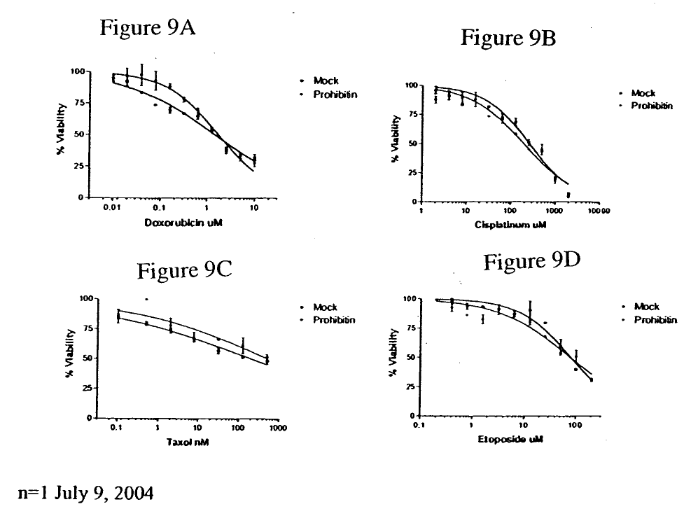

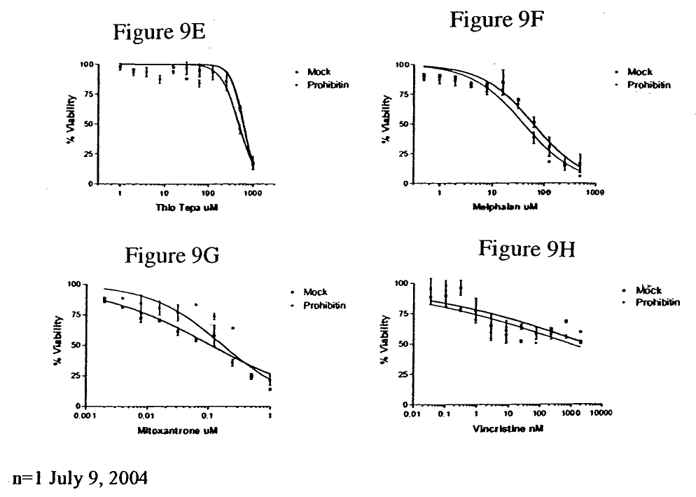

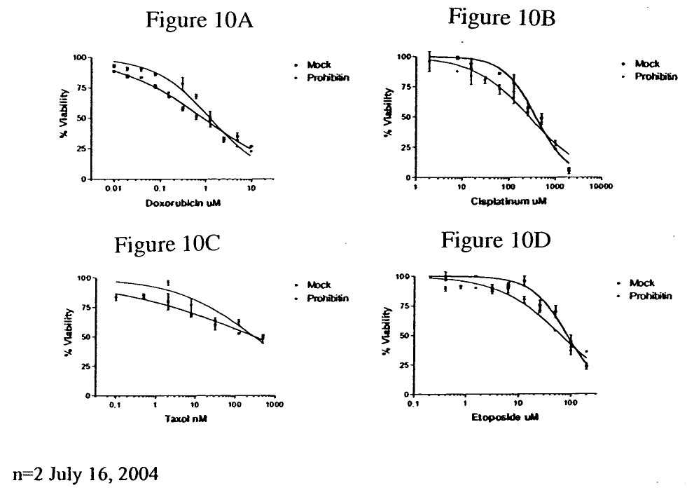

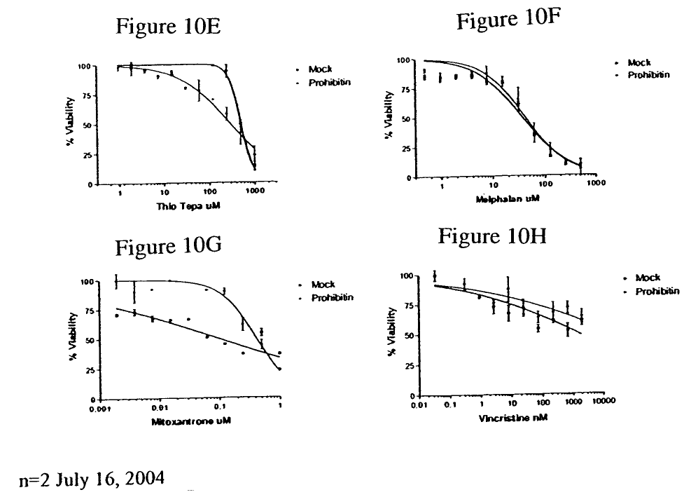

MTT cytotoxicity assays were performed on cell lines treated with chemotherapeutic drugs and prohibitin siRNA. MDA cell lines treated with prohibitin siRNA showed increased sensitivity to cisplatinum, thiotepa, vincristin, and melphalan as compared to mock-treated cells (FIG. 9A-9H). In additional experiments, MDA cell lines were more sensitive to cisplatinum, etoposide, and thiotepa as compared to controls (FIGS. 10A-10H).

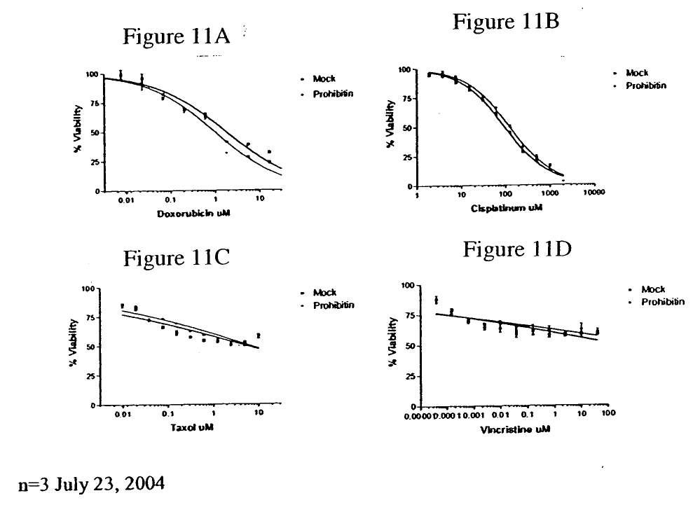

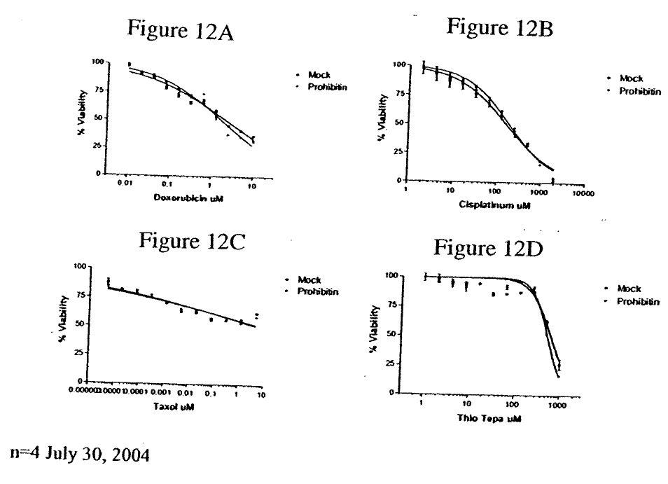

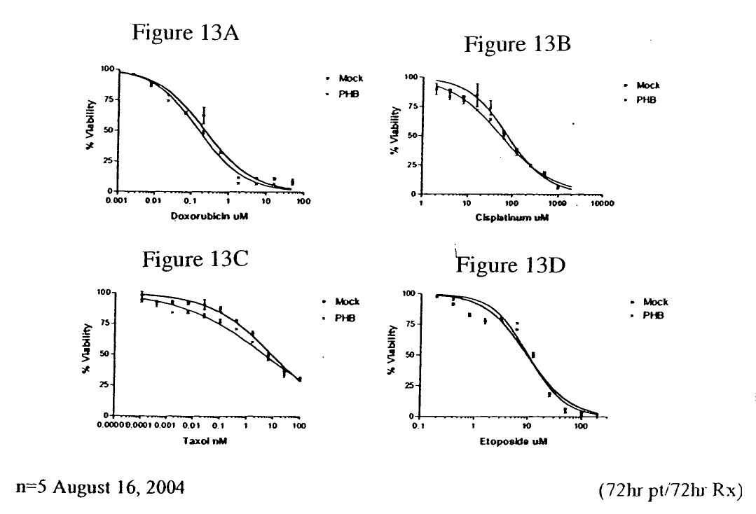

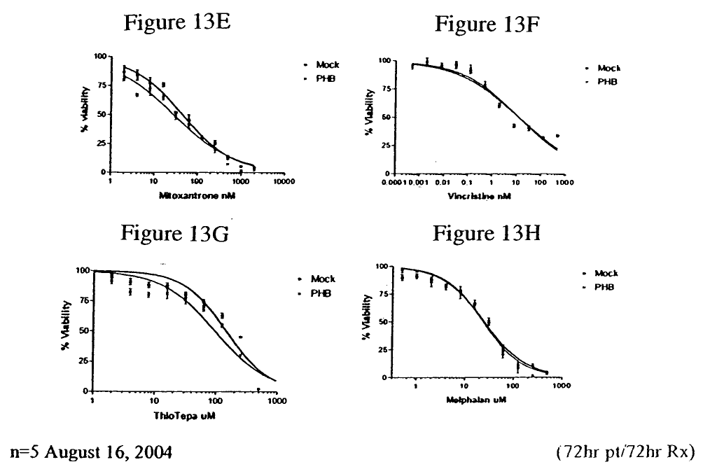

MDA cell lines also showed increased sensitivity to doxorubicin when treated with prohibitin siRNA as compared to controls (FIG. 11A). These results were confirmed in subsequent experiments in which MDA cells also showed increased sensitivity to taxol and thiotepa as compared to mock controls (FIGS. 12A-12D). Additional experiments were performed using prohibitin siRNA to confirm the results obtained in previous experiments (FIGS. 13A-13H). These experiments again confirmed results obtained previously in which MDA cells treated with prohibitin siRNA showed increased sensitivity to doxorubicin and cisplatinum as compared to mock controls (FIGS. 13A-13B). These experiments also showed that prohibitin siRNA treatment increased the sensitivity of MDA cells to the chemotherapeutic drugs taxol, mitoxantrone, and thiotepa as compared to mock controls (FIGS. 13C, 13E, and 13G).

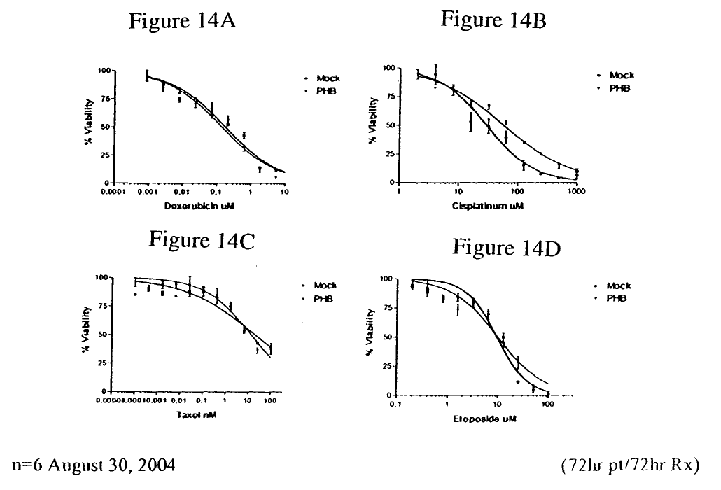

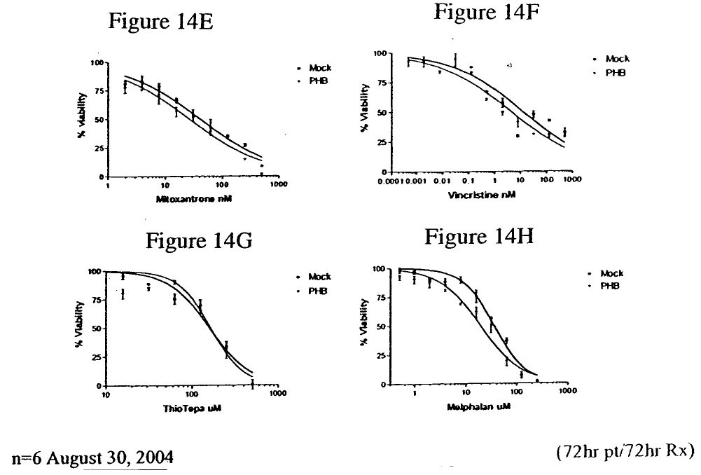

MDA cells were subjected to prohibitin siRNA treatment to further confirm the results obtained previously. MDA cells treated with prohibitin siRNA were more sensitive to doxorubicin, taxol, mitoxantrone, vincristin, and melphalan treatments as compared to mock controls (FIGS. 14A, 14C, 14E, 14F, and 14H). These results and the results shown above establish that prohibitin siRNA treatment improves the efficacy of several chemotherapeutic drugs.

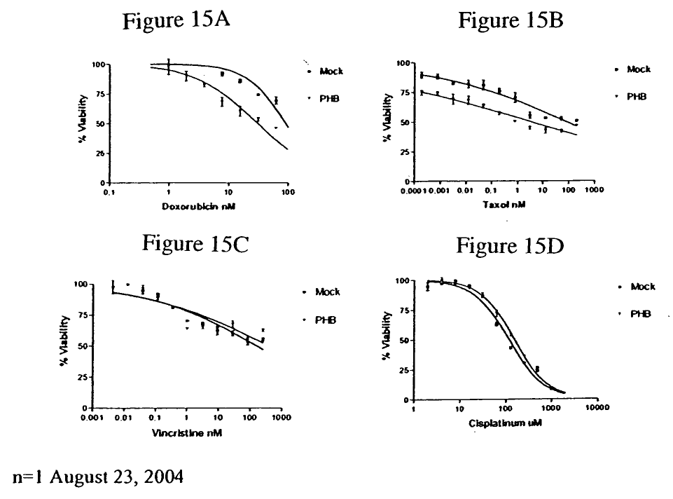

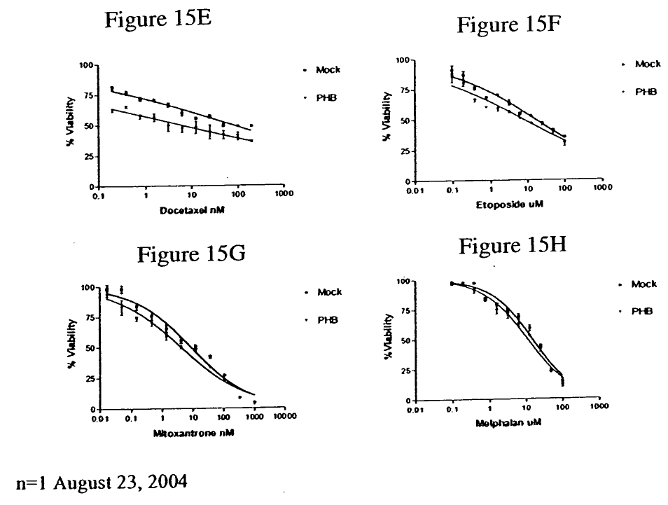

In addition to the tests performed on MDA cell lines, MCF-7 cell lines were treated with prohibitin siRNA and chemotherapeutic drugs. MCF-7 cells treated with prohibitin siRNA were more sensitive to doxorubicin, taxol, etoposide, mitoxantrone, docetaxel, and melphalan (FIGS. 15A-15H). The MCF-7 cells treated with prohibitin siRNA showed the most striking sensitivity to taxol (FIG. 15B). Sensitivity to taxol increased by 30 fold as compared to controls when MCF-7 cells were treated with prohibitin siRNA. These results establish that prohibitin siRNA improved the efficacy of chemotherapeutic drugs in breast adenocarcinoma cell lines.

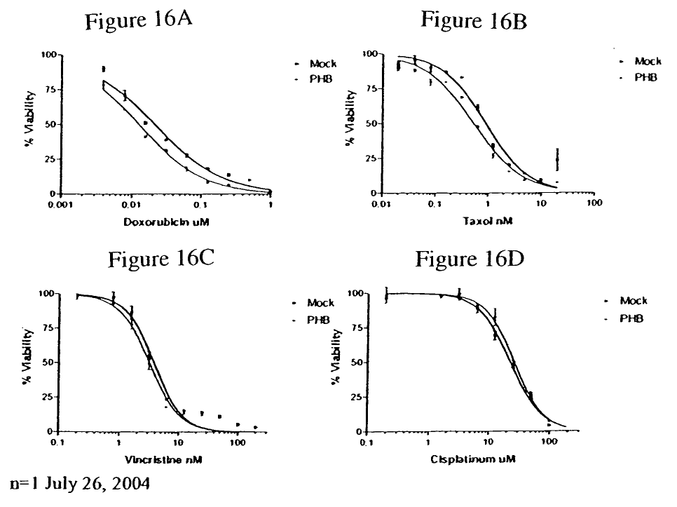

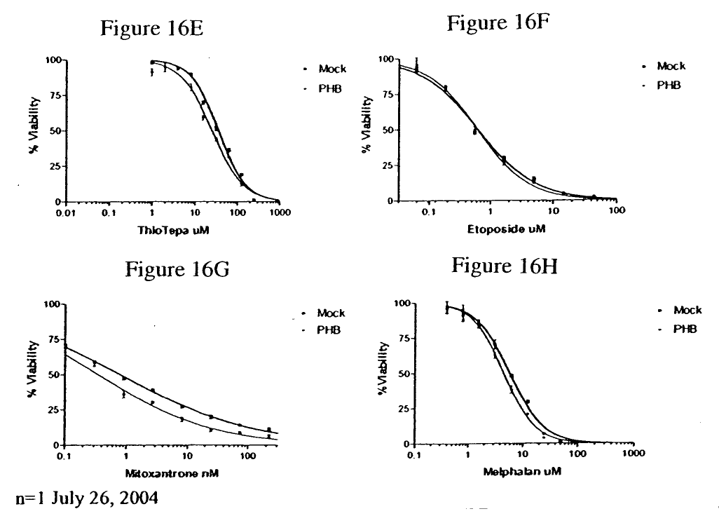

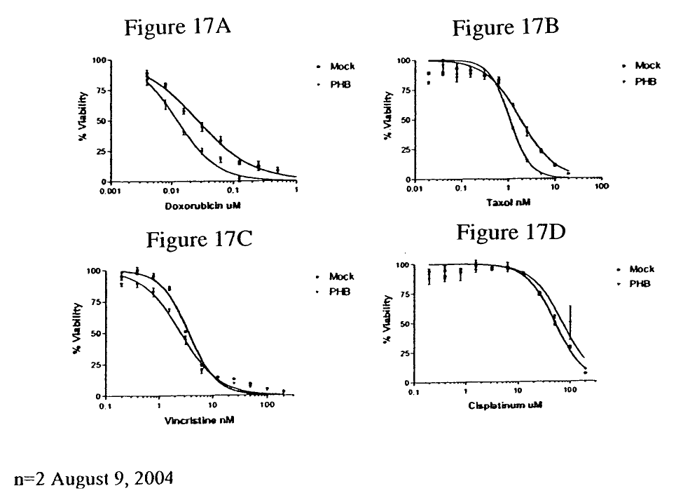

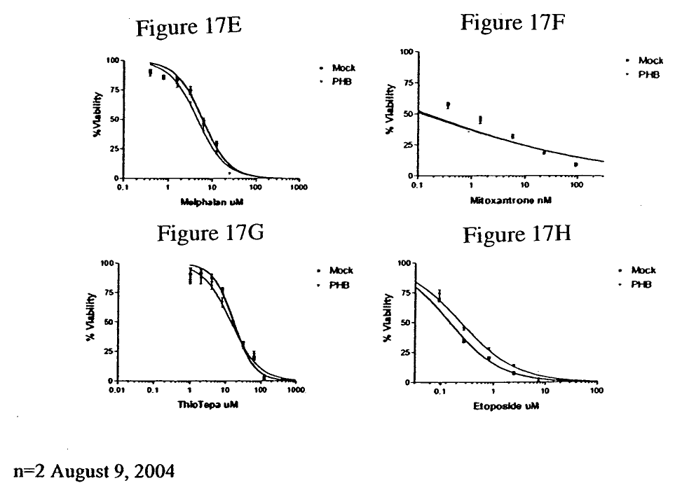

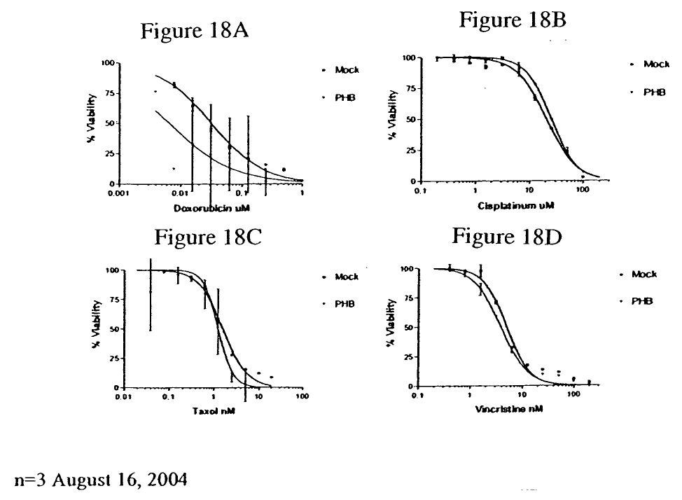

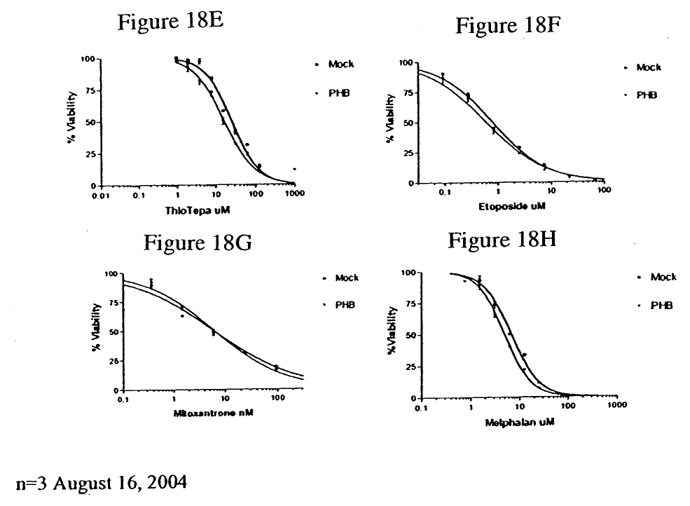

The ovarian cancer cell line SKOV3 was treated with prohibitin siRNA in combination with various chemotherapeutic drugs to determine whether prohibitin silencing affects the chemosensitivity of the cells. The first set of experiments established that most chemotherapeutic drugs were more effective when accompanied by prohibitin siRNA treatment (FIGS. 16A-16H). These results were confirmed by additional testing on the SKOV3 cell line (FIGS. 17A-17H). Further studies showed that most chemotherapeutic drugs were more effective against SKOV3 cells when the cells were treated with prohibitin siRNA as compared to cells treated with mock treatments (FIGS. 18A-18H).

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Overexpression of a 30 kD Protein in Cancer Cell Lines

Studies were performed to determine what proteins, if any, were differentially expressed in chemotherapeutic drug-resistant tumor cell lines as compared to their drug-sensitive counterparts. The nine different cell lines used in the Examples below are listed in Table 2.

TABLE 2

| Drug-Sensitive Cell Lines | Drug-Resistant Cell Lines |
|---|---|
| MCF-7 | MCF-7/AR |
| SKOV3 | MCF-7/VLB |
| 2008 | MCF-7/VCR |
| H69 | MCF-7/Mito |
| HS578T | MCF-7/Taxol |
| BT549 | MDA/taxol |
| HeLa | MDA-MB-231/AR |
| | MDA/Mito |
| | SKOV3/DOXO |
| | SKOV3/Taxol |
| | SKOV3/VLB |
| | 2008/DOXO |
| | 2008/Taxol |
| | 2008/CIS |
| | H69/AR |

Drug-sensitive control cell lines were obtained from were obtained from ATCC (Manassas, Va., USA). MCF7/AR was obtained from McGill University, Montreal, Qc, Canada. MDA-MB-231/AR was derived at Aurelium BioPharma Inc. (Montreal, QC, Canada). Additional chemotherapeutic drug-resistant cell lines used in the experiments were derived from a drug-sensitive clone of the “parent” cancer cell line representing a particular tissue.

All cell culture materials and reagents were obtained from Gibco Life Technologies (Burlington, Ont., Canada), or Sigma Chemical Corp. (St. Louis, Mo., USA) unless otherwise indicated.

Cells were cultured in a MEM medium supplemented with 10% fetal bovine serum (MCF7 and derivatives) or in DMEM high glucose medium supplemented with 10% fetal bovine serum (MDA-MB-231 and derivatives). All culture media contained L-glutamine (final concentration of 2 mM). The cells were grown in the absence of antibiotics at 37° C. in a humid atmosphere of 5% CO₂ and 95% air. Chemotherapeutic drug-resistant cells (MCF-7/AR and MDA-MB-231/AR) were grown continuously with appropriate concentrations of cytotoxic drugs. All cell lines were examined for and determined to be free of mycoplasma contamination using a PCR-based mycoplasma detection kit according to manufacturer's instructions (Stratagene Inc., San Diego, Calif., USA). Chemotherapeutic drug-resistant cell lines were routinely tested for chemotherapeutic drug resistance using a panel of different drugs representing different classes.

Cell extracts from drug-resistant and drug-sensitive cell lines were prepared to determine the expression levels of potential therapeutic targets in drug-resistant cells. Briefly, cultured cells were rinsed 2 times with 15 ml of 1× phosphate buffered saline (“PBS”), and harvested by trypsinization. Cells were collected in a 15 ml tube by centrifugation at 1000 rpm for 5 min. The supernatant was discarded and cells were washed 3 times with 1×PBS. The cell pellet was transferred to an Eppendorf tube and 500 ml of 1×PBS were added. Cells were centrifuged 5 min. at 3000 rpm in an Eppendorf Microfuge. The supernatant was removed and cells were then lysed in 50 ml-150 ml of lysis buffer (50 mM Tris, pH 7.5, 150 mM NaCl, 1% NP-40, 0.5% sodium deoxycholate), containing protease inhibitors (1 mg/ml pepstatin, 1 mg/ml leupeptin, 1 mg/ml benzamidine, 0.2 mM PMSF) and incubated 5 min. on ice. The cell lysates were then centrifuged at 14,000×g for 10 min. at 4° C. The protein concentration of the supernatants was determined by the DC Protein assay (BioRad, Hercules, Calif.). Samples were subsequently stored at −80° C. until ready for analysis.

Total cell lysates were thawed and then incubated with 1 U/ml DNAse I (New England BioLabs, Inc., Beverly, Mass.), 5 mM MgCl₂ (final concentration) for 2 hours on ice. Their protein concentration was determined using the RC DC protein assay kit from BIORAD according to manufacturer's instructions (BioRad Laboratories, Hercules, Calif.) (see also Lowry et al., (1951) J. Biol. Chem. 193: 265-275). Equivalent amounts of proteins (250 mg) from total cell extracts from sensitive (MCF7, MDA-MB-231) and chemotherapeutic drug-resistant cells (MCF7/AR and MDA-MB-231/AR) were analyzed by polyacrylamide gel electrophoresis followed by blotting to a nitrocellulose membrane. The membrane was subsequently contacted with an anti-prohibitin antibody obtained from Abcam, Inc., Cambridge, Mass. The immunoblots were incubated with an anti-IgG secondary antibody and visualized using horseradish peroxidase using the manufacturer's protocol (Bio-Rad Laboratories, Hercules, Calif.).

The experiments identified a 30 kD protein that was of similar size to prohibitin, and had significant reactivity with anti-prohibitin antibody (FIGS. 1A-1C).

### Example 2

### Targeted Silencing of Prohibitin in MDA Breast Cancer Cell Lines

To establish the importance of prohibitin to the expression of the drug-resistant phenotype in MDA cell lines, prohibitin expression was silenced using RNAi. Briefly, the following siRNA duplexes targeting the human prohibitin mRNA were designed and purchased either from Ambion (Austin, Tex.) or Invitrogen (Carlsbad, Calif.). The siRNA duplex sequences corresponding to nucleotides 425 through 443 (GenBank SEQ ID NUMBER: U49725.1) targeting the start of the prohibitin mRNA transcript were:

sense strand 5′-GGAAGUGGUCUGUCUGUUAtt-3′ (SEQ ID NO: 2); and

antisense strand: 5′-UAACAGACAGACCACUUCCtt-3′ (SEQ ID NO: 1).

The siRNA duplex was predesigned, synthesized with 3′TT overhangs, purified and annealed by Ambion (Austin, Tex.). To monitor transfection efficiency, a Cy3-labeled GL2 siRNA duplex against firefly luciferase was purchased from Dharmacon, Inc. (Chicago, Ill.). For the chemically modified Stealth siRNA's, the non-targeting siGLO™ fluorescent siRNA duplex (Dharmacon, Chicago, Ill.) or the Block-it™ Fluorescent oligonucleotide (Invitrogen, Carlsbad, Calif.) was used. Transfection efficiencies were typically evaluated 24-48 hrs post transfection using a fluorescence microscope. The levels achieved were routinely greater than 95%.

For a typical siRNA transfection, 1 nmole of the annealed siRNA duplex was mixed with 1.4 ml of Opti-MEM reagent (Invitrogen). In another tube, 85 ml of Oligofectamine reagent (Invitrogen, Carlsbad, Calif.) was mixed with 600 ml of Opti-MEM. The two solutions were combined and mixed gently by inversion and incubated for 20 min. at RT. The resulting solution was added to the cultured cells drop by drop in a 10 cm dish (cells are approximately 40-50% confluent). The next day the transfected cells were trypsinized and seeded in 6 or 96-well plates and further incubated for the indicated amount of time (assay dependent) before further analysis. Immunoblots depicting the results of prohibitin siRNA on prohibitin expression are shown in FIGS. 2-5.

### Example 3

### Effects of Prohibitin Silencing on MDA Breast Cancer Cell Survival

1. MTT Cytotoxicity Assay

Cell survival was determined using the MTT cytotoxicity assay (see, e.g., Tokuyama et al. (2005) Anticancer Res. 25(1A): 17-22). RNA-transfected cells were seeded in triplicate into 96-well plates at 5×10³ cells/well 48 hrs post-transfection. The cells were incubated for an additional 16 to 24 hrs before they were exposed to increasing concentrations of cytotoxic drugs. Doxorubicin (adriamycin), cisplatinum, taxol, vinblastin, vincristin, and mitoxantrone were all purchased from Sigma Corp. (St. Louis, Mo.). Stocks were made as follows: 6 mM for doxorubicin, 1.1 mM for vincristin and vinblastin; 1.1 mM for taxol, 50 mM for cisplatinum both in DMSO; and 0.97 mM mitoxantrone in ethanol. Appropriate dilutions were made in the respective media for each cell line. Following addition of drugs, incubation was continued for an additional 72 hrs. Twenty-five ml of MTT dye (5 mg/ml) were added into each well and the plate was further incubated at 37° C. for 4 hrs. The dye was solubilized with 10% Triton X-100, 0.01 N HCl and further incubated at 37° C. in the dark for 30 min. Cell viability was determined by measure of absorption at 570 nm in a Wallac multiwell plate reader (PerkinElmer, Inc., Boston, Mass.). The averages of triplicate wells were plotted using the Prism software (GraphPad Software, Inc., San Diego, Calif.).

The results indicate that MDA cells generally had decreased viability when treated with chemotherapeutic drugs in combination with prohibitin siRNA (FIGS. 9-14). The results are further summarized in Tables 3-8. Each table below summarizes the results of an individual experiment performed in triplicate. The EC₅₀ results were obtained 72 hours post transfection with either a vector expressing prohibitin-encoding RNA or a mock vector.

TABLE 3
Transfection of MDA Breast Cancer Cell
Line With Vector Expressing Prohibitin

| Chemotherapeutic Drug | Control | Prohibitin |
|---|---|---|
| Doxorubicin (nM) | 1.458 (R2 = 0.9532) | 1.734 (R2 = 0.9239) |
| | | 1.2x IR |
| Taxol (nM) | 199.9 (R2 = 0.9382) | 514.6 (R2 = 0.7017) |
| | | 2.6x IR |
| Cisplatinum (μM) | 255.1 (R2 = 0.9232) | 189.1 (R2 = 0.9270) |
| | | 1.2x IS |
| Etoposide (μM) | 78.85 (R2 = 0.9469) | 76.54 (R2 = 0.9099) |
| | | NC |
| Thiotepa (μM) | 600.0 (R2 = 0.9264) | 475.1 (R2 = 0.9044) |
| | | 1.3x IS |
| Vincristin (nM)) | 2845 (R2 = 0.4598) | 837.1 (R2 = 0.5137) |
| | | 3.4x IS |
| Mitoxantrone (nM) | 0.1103 (R2 = 0.8395) | 0.1731 (R2 = 0.8816) |
| | | X1.6 IR |
| Melphalan (μM) | 60.12 (R2 = 0.9013) | 39.05 (R2 = 0.9145) |
| | | 1.5x IS |

IS: “increased sensitivity” to the particular drug
IR: “increased in resistance” to the drug
NC: “no change”
R2: “statistical fit to the curve”

TABLE 4
Transfection of MDA Breast Cancer Cell
Line With Vector Expressing Prohibitin

| Chemotherapeutic | | |
|---|---|---|
| Drug | Control | Prohibitin |
| Doxorubicin (nM) | 0.8734 (R2 = 0.9526) | 1.252 (R2 = 0.9457) |
| | | 1.4x IR |
| Taxol (nM) | 316.1 (R2 = 0.8788) | 301.3 (R2 = 0.8370) |
| | | NC |
| Cisplatinum (μM) | 373.8 (R2 = 0.9372) | 271.1 (R2 = 0.9079) |
| | | 1.4x IS |
| Etoposide (μM) | 83.87 (R2 = 0.9378) | 63.03 (R2 = 0.9342) |
| | | 1.3x IS |
| Thiotepa (μM) | 510.3 (R2 = 0.9342) | 291.2 (R2 = 0.9158) |
| | | 1.7x IS |
| Vincristin (nM)) | 1485 (R2 = 0.6505) | 18613 (R2 = 0.4918) |
| | | 12.5x IR |
| Mitoxantrone (nM) | 0.09607 (R2 = 0.7862) | 0.4090 (R2 = 0.9464) |
| | | 4.2x IR |
| Melphalan (μM) | 36.14 (R2 = 0.9264) | 41.04 (R2 = 0.9070) |
| | | 1.1x IR |

IS: “increased sensitivity” to the particular drug
IR: “increased in resistance” to the drug
NC: “no change”
R2: “statistical fit to the curve”

TABLE 5
Transfection of MDA Breast Cancer Cell
Line With Vector Expressing Prohibitin

| Chemotherapeutic Drug | Control | Prohibitin |
|---|---|---|
| Doxorubicin (nM) | 1.749 (R2 = 0.9364) | 0.9934 (R2 = 0.9652) |
| | | 1.8x IS |
| Taxol (nM) | 5.11 (R2 = 0.7195) | 6.548 (R2 = 0.8329) |
| | | 1.3x IR |
| Cisplatinum (μM) | 94.32 (R2 = 0.9898) | 123.8 (R2 = 0.9916) |
| | | 1.3x IR |
| Vinblastin (nM) | 156.5 (R2 = 0.5946) | 2552 (R2 = 0.5887) |
| | | NA |

IS: “increased sensitivity” to the particular drug
IR: “increased in resistance” to the drug
NC: “no change”
R2: “statistical fit to the curve”

TABLE 6
Transfection of MDA Breast Cancer Cell
Line With Vector Expressing Prohibitin

| Chemotherapeutic Drug | Control | Prohibitin |
|---|---|---|
| Doxorubicin (nM) | 2.246 (R2 = 0.9586) | 1.717 (R2 = 0.9600) |
| | | 1.3x IS |
| Taxol (nM) | 10.77 (R2 = 0.7827) | 6.193 (R2 = 0.8803) |
| | | 1.7x IS |
| Cisplatinum (μM) | 163.7 (R2 = 0.9545) | 189.4 (R2 = 0.9812) |
| | | 1.1x IR |
| Thiotepa (μM) | 628.1 (R2 = 0.8994) | 526.6 (R2 = 0.9239) |
| | | 1.2x IS |

IS: “increased sensitivity” to the particular drug
IR: “increased in resistance” to the drug
NC: “no change”
R2: “statistical fit to the curve”

TABLE 7
Transfection of MDA Breast Cancer Cell
Line With Vector Expressing Prohibitin

| Chemotherapeutic Drug | Control | Prohibitin |
|---|---|---|
| Doxorubicin (nM) | 0.2317 (R2 = 0.9573) | 0.1597 (R2 = 0.9828) |
| | | 1.4x IS |
| Cisplatinum (μM) | 76.81 (R2 = 0.9603) | 54.48 (R2 = 0.9774) |
| | | 1.4x IS |
| Taxol (nM) | 8.322 (R2 = 0.9472) | 4.381 (R2 = 0.9707) |
| | | 1.9x IS |
| Etoposide (μM) | 9.559 (R2 = 0.9596) | 10.01 (R2 = 0.9564) |
| | | NC |
| Mitoxantrone (nM) | 44.06 (R2 = 0.9638) | 27.64 (R2 = 0.9469) |
| | | 1.6x IS |
| Thiotepa (μM) | 146.2 (R2 = 0.9352) | 97.1 (R2 = 0.9075) |
| | | 1.5x IS |
| Vincristin (nM) | 12.28 (R2 = 0.9295) | 12.28 (R2 = 0.9156) |
| | | NC |
| Melphalan (μM) | 24.85 (R2 = 0.9718) | 23.85 (R2 = 0.9586) |
| | | NC |

IS: “increased sensitivity” to the particular drug
IR: “increased in resistance” to the drug
NC: “no change”
R2: “statistical fit to the curve”

TABLE 8
Transfection of MDA Breast Cancer Cell
Line With Vector Expressing Prohibitin

| Chemotherapeutic Drug | Control | Prohibitin |
|---|---|---|
| Doxorubicin (nM) | 0.1771 (R2 = 0.9743) | 0.1389 (R2 = 0.9489) |
| | | 1.3x IS |
| Cisplatinum (μM) | 29.31 (R2 = 0.9171) | 57.04 (R2 = 0.9717) |
| | | 1.9x IR |
| Taxol (nM) | 19.21 (R2 = 0.8261) | 12.66 (R2 = 0.9064) |
| | | 1.5x IS |
| Etoposide (μM) | 9.308 (R2 = 0.9502) | 9.645 (R2 = 0.9159) |
| | | NC |
| Mitoxantrone (nM) | 40.94 (R2 = 0.9388) | 26.59 (R2 = 0.9003) |
| | | 1.5x IS |
| Thiotepa (μM) | 158.9 (R2 = 0.9363) | 164.1 (R2 = 0.9049) |
| | | NC |
| Vincristin (nM) | 12.4 (R2 = 0.7592) | 4.297 (R2 = 0.9096) |
| | | 2.9x IS |
| Melphalan (μM) | 35.4 (R2 = 0.9689) | 19.14 (R2 = 0.9360) |
| | | 1.8x IS |

IS: “increased sensitivity” to the particular drug
IR: “increased in resistance” to the drug
NC: “no change”
R2: “statistical fit to the curve”

### Example 4

### Targeted Silencing of Prohibitin in MCF-7 Breast Cancer Cell Lines

Targeted silencing of prohibitin was performed as described in Example 2 above. Immunoblots depicting the results of prohibitin siRNA on prohibitin expression are shown in FIG. 6.

### Example 5

Effects of Prohibitin Silencing on MCF-7 Breast Cancer Cell Survival

Cell survival was determined as described in Example 3 above. The results of MTT cytotoxicity assays are shown in FIG. 15. The results are further summarized in Table 9.

TABLE 9
Transfection of MCF-7 Breast Cancer Cell
Line With Vector Expressing Prohibitin

| Chemotherapeutic Drug | Control | Prohibitin |
|---|---|---|
| Doxorubicin (nM) | 90.64 (R2 = 0.9665) | 29.96 (R2 = 0.9521) |
| | | 3.0x IS |
| Taxol (nM) | 78.4 (R2 = 0.8825) | 2.616 (R2 = 0.8573) |
| | | 30.0x IS |
| Cisplatinum (μM) | 117.7 (R2 = 0.9815) | 151.0 (R2 = 0.9901) |
| | | 1.3x IR |
| Etoposide (μM) | 16.11 (R2 = 0.9443) | 7.275 (R2 = 0.9162) |
| | | 2.2x IS |
| Mitoxantrone (nM) | 8.682 (R2 = 0.9616) | 3.939 (R2 = 0.9578) |
| | | 2.2x IS |
| Vincristin (nM) | 163.3 (R2 = 0.8647) | 350.2 (r2 = 0.7098) |
| | | 2.1x IR |
| Docetaxel (nM) | 73.49 (R2 = 0.9195) | 5.342 (R2 = 0.7369) |
| | | 13.8x IS |
| Melphalan (μM) | 14.7 (R2 = 0.9705) | 10.53 (R2 = 0.9724) |
| | | 1.4x IS |

IS: “increased sensitivity” to the particular drug
IR: “increased in resistance” to the drug
NC: “no change”
R2: “statistical fit to the curve”

### Example 6

### Targeted Silencing of Prohibitin in SKOV3 Ovarian Cancer Cell Lines

Targeted silencing of prohibitin was performed as described in Example 2 above. Immunoblots depicting the results of prohibitin siRNA on prohibitin expression are shown in FIGS. 7-8.

### Example 7

### Effects of Prohibitin Silencing on SKOV3 Ovarian Cancer Cell Survival

Cell survival was determined as described in Example 3 above. The results of MTT cytotoxicity assays are shown in FIGS. 16-18. The results are also summarized in Tables 10 and 11. Each table summarizes the results of an individual experiment performed in triplicate.

TABLE 10
Transfection of SKOV3 Ovarian Cancer Cell
Line With Vector Expressing Prohibitin

| Chemotherapeutic Drug | Control | Prohibitin |
|---|---|---|
| Doxorubicin (nM) | 21.28 (R2 = 0.9777) | 12.8 (R2 = 0.9628) |
| | | 1.7x IS |
| Taxol (nM) | 0.8947 (R2 = 0.9377) | 0.5358 (R2 = 0.9791) |
| | | 1.7x IS |
| Cisplatinum (μM) | 22.43 (R2 = 0.9875) | 26.31 (R2 = 0.976) |
| | | 1.2x IR |
| Etoposide (μM) | 0.6206 (R2 = 0.9907) | 0.6114 (R2 = 0.9885) |
| | | NC |
| Thiotepa (μM) | 34.98 (R2 = 0.9872) | 24.95 (R2 = 0.9855) |
| | | 1.4x IS |
| Vincristin (nM) | 3.754 (R2 = 0.9698) | 3.168 (R2 = 0.9729) |
| | | 1.2x IS |
| Mitoxantrone (nM) | 0.8012 (R2 = 0.9639) | 0.3578 (R2 = 0.9716) |
| | | 2.2x IS |
| Melphalan (μM) | 5.79 (R2 = 0.9885) | 4.536 (R2 = 0.9912) |
| | | 1.3x IS |

IS: “increased sensitivity” to the particular drug
IR: “increased in resistance” to the drug
NC: “no change”
R2: “statistical fit to the curve”

TABLE 11
Transfection of SKOV3 Ovarian Cancer Cell
Line With Vector Expressing Prohibitin

| Chemothera- | | |
|---|---|---|
| peutic Drug | Control | Prohibitin |
| Doxorubicin | 0.02652 (R2 = 0.9653) | 0.01231 (R2 = 0.9225) |
| (nM) | | 2.2x IS |
| Taxol (nM) | 1.824 (R2 = 0.9655) | 1.086 (R2 = 0.9421) |
| | | 1.7x IS |
| Cisplatinum | 49.99 (R2 = 0.9768) | 68.83 (R2 = 0.816) |
| (μM) | | 1.4x IR |
| Etoposide | 0.148 (R2 = 0.9263) | 0.2408 (R2 = 0.9744) |
| (μM) | | 1.6x IR |
| Thiotepa | 16.47 (R2 = 0.9255) | 13.92 (R2 = 0.9567) |
| (μM) | | 1.2x IS |
| Vincristin | 3.581 (R2 = 0.9785) | 2.531 (R2 = 0.9832) |
| (nM) | | 1.4x IS |
| Mitoxantrone | 0.1468 (R2 = 0.8342) | 0.1179 (R2 = 0.8263) |
| (nM) | | 1.2x IS |
| Melphalan | 5.946 (R2 = 0.9719) | 4.565 (R2 = 0.972) |
| (μM) | | 1.3x IS |

IS: “increased sensitivity” to the particular drug
IR: “increased in resistance” to the drug
NC: “no change”
R2: “statistical fit to the curve”

### Example 8

### Prohibitin-Targeted Therapy Against Hematological Cancer Cells

1. Treatment of MDR Hematological Cancer Cells

In order to determine whether targeting prohibitin is useful in treating a preexisting cancerous condition, MHC-matched mice, 5 to 7 weeks old, receive a subcutaneous (s.c.) injection of the cells 5×10⁵ hematological tumor cells, and tumors are allowed to form. Tumor growth starting on the first day of treatment is measured by palpitation, and the volume of the xenograft is monitored every 4 days. Tumors are allowed to grow to a sufficient size (5.5 mm) for appropriate analysis of the effects of prohibitin treatment on tumor sensitivity to chemotherapeutic drugs. Mice are then treated with a prohibitin siRNA (3 μg daily for 16 days) designed to decrease the level of expression of prohibitin. Control mice receive no treatment, treatment with taxol or doxorubicin alone (4 mg/kg daily) or treatment with control siRNA sequences that are not complementary to murine prohibitin mRNA (3 μg daily for 16 days for each treatment) in combination with taxol or doxorubicin (4 mg/kg daily). Taxol and doxorubicin can be obtained commercially from Sigma Corp. (St. Louis, Mo.).

Treatment with siRNA specific for prohibitin mRNA sequences increases the sensitivity of hematological tumors to chemotherapeutic drug treatment regimes. As a result, the mice that receive the composition show a better prognosis (i.e., smaller tumor or fewer tumor cells) as compared to mice that receive only the targeting agent or only the taxol or doxorubicin.

Control siRNA sequences are utilized that do not represent binding sequences to murine prohibitin (3 μg daily for 16 days for each treatment). The animal's weight is measured every 4 days. Tumor growth starting on the first day of treatment is measured by palpitation and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the prohibitin siRNA and chemotherapeutic drugs are compared to tumor weights from mice treated with control siRNA and chemotherapeutic drugs. Tumor cell count is determined by trypsinizing tumors in DMEM medium supplemented with 10% fetal bovine serum until cells are in free suspension. Cells are then transferred to 6-well plates for counting. Cell counts are compared. All experiments are performed in triplicate.

2. Treatment of Mammary Adenocarcinoma

In further studies, the efficacy of a prohibitin-targeted therapeutic in treating mammary adenocarcinoma cells (MCF-7/AR) is assessed. Briefly, male thymic nude mice 5 to 7 weeks old, weighing 18 g to 22 g, are used for the MCF-7/ADR xenografts. Mice receive a subcutaneous (s.c.) injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, prohibitin siRNA alone (3 μg daily for 16 days), or both taxol and prohibitin siRNA (3 μg daily for 16 days for each treatment). Control siRNA sequences are utilized that do not represent binding sequences to murine prohibitin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the prohibitin siRNA and chemotherapeutic drugs are compared to tumor weights from mice treated with control siRNA and chemotherapeutic drugs. Cell counts are compared. All experiments are performed in triplicate.

Mice treated with the prohibitin siRNA have smaller tumors by weight than mice treated with control siRNA. In addition, total cell numbers of tumors isolated from mice treated with prohibitin siRNA are lower than mice treated with control siRNA.

### Example 9

Prohibitin Targeted Therapy Against Hematological Cancer Cells

1. Treatment of Hematological Tumors

In order to determine whether targeting prohibitin is useful in treating a preexisting cancerous condition, MHC-matched mice, 5 to 7 weeks old, receive an s.c. injection of the cells 5×10⁵ hematological tumor cells, and tumors are allowed to form. Tumor growth starting on the first day of treatment is measured by palpitation and the volume of the xenograft is monitored every 4 days. Tumors are allowed to grow to a sufficient size (5.5 mm) for appropriate analysis of the effects of prohibitin treatment on tumor sensitivity to chemotherapeutic drugs. Mice are then treated with a prohibitin siRNA (3 μg daily for 16 days) designed to decrease the level of expression of prohibitin. Control mice receive no treatment, treatment with taxol or doxorubicin alone (4 mg/kg daily) or treatment with control siRNA sequences that are not complementary to murine prohibitin mRNA (3 μg daily for 16 days for each treatment) in combination with taxol or doxorubicin (4 mg/kg daily). Taxol and doxorubicin can be obtained commercially from Sigma Corp. (St. Louis, Mo.).

Control siRNA sequences are utilized that do not represent binding sequences to murine prohibitin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the prohibitin siRNA and chemotherapeutic drugs are compared to tumor weights from mice treated with control siRNA and chemotherapeutic drugs. Tumor cell count is determined by trypsinizing tumors in DMEM medium supplemented with 10% fetal bovine serum until cells are in free suspension. Cells are then transferred to 6-well plates for counting. Cell counts are compared. All experiments are performed in triplicate.

2. Treatment of Mammary Adenocarcinoma

In further studies, the efficacy of a prohibitin-targeted therapeutic in treating a mammary adenocarcinoma cells (MCF-7) is assessed. Briefly, male thymic nude mice 5 to 7 weeks old, weighing 18 g to 22 g, are used for the MCF-7/ADR xenografts. Mice receive an s.c. injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, prohibitin siRNA alone (3 μg daily for 16 days), or both taxol and prohibitin siRNA (3 μg daily for 16 days for each treatment). Control siRNA sequences are utilized that do not represent binding sequences to murine prohibitin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the prohibitin siRNA and chemotherapeutic drugs are compared to tumor weights from mice treated with control siRNA and chemotherapeutic drugs.

Mice treated with the prohibitin siRNA have smaller tumors by weight than mice treated with control siRNA. In addition, total cell number in tumors isolated from mice treated with prohibitin siRNA is lower than mice treated with control siRNA.

### Example 10

### Prohibitin Liposome Formulation for Targeted Therapy Against Hematological Cancer Cells

1. Treatment of Hematological Cancer

In order to determine whether targeting prohibitin is useful in treating a preexisting cancerous condition, MHC-matched mice, 5 to 7 weeks old, receive an s.c. injection of the cells 5×10⁵ hematological tumor cells, and tumors are allowed to form. Tumor growth starting on the first day of treatment is measured by palpitation and the volume of the xenograft is monitored every 4 days. Tumors are allowed to grow to a sufficient size (5.5 mm) for appropriate analysis of the effects of prohibitin treatment on tumor sensitivity to chemotherapeutic drugs. Mice are then treated with a liposome formulation containing prohibitin siRNA designed to decrease the level of expression of prohibitin.

Liposome formulations are produced as described previously (Shi et al. (2000) Proc. Natl. Acad. Sci. USA. 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and are sonicated for 10 min. Prohibitin siRNA, at a concentration of between 5 μg/ml and 10 μg/ml, is then added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

The liposome treatment introduces 3 μg of prohibitin-targeted siRNA per day for 16 days. Control mice receive no treatment, treatment with taxol or doxorubicin alone (4 mg/kg daily) or treatment with liposomes containing control siRNA sequences that are not complementary to murine prohibitin mRNA (3 μg daily for 16 days for each treatment) in combination with taxol or doxorubicin (4 mg/kg daily). Taxol and doxorubicin can be obtained commercially from Sigma Corp. (St. Louis, Mo.).

The cancer cells treated with liposome/prohibitin siRNA treatment show an increase in sensitivity to chemotherapeutic treatment regimes. As a result, the mice that receive the composition show a better prognosis (i.e., smaller tumor or fewer tumor cells) as compared to mice that receive only the targeting agent or only the vincristin.

A determination of decreased tumor size or cancer cell number is made by sacrificing the mice and excising the tumor. The size of the tumor in mice treated with the prohibitin targeting agent and chemotherapy is measured and compared to measurements obtained from tumors in mice treated with chemotherapy alone. Tumor cell count is determined by trypsinizing tumors in DMEM medium supplemented with 10% fetal bovine serum until cells are in free suspension. Cells are then transferred to six well plates for counting. Cell counts are compared. All experiments are performed in triplicate.

2. Treatment of Mammary Adenocarcinoma

In further studies, the efficacy of a prohibitin-targeted therapeutic in treating a mammary adenocarcinoma cells (MCF-7) is assessed. Briefly, male thymic nude mice 5 to 7 weeks old, weighing 18 g to 22 g, are used for the MCF-7/ADR xenografts. Mice receive an s.c. injection of the cells using 5×1 cells/inoculation under the shoulder.

Liposome formulations are produced as described previously (Shi et al. (2000) Proc. Natl. Acad. Sci. USA. 97(13): 7567-7572). Briefly, POPC (19.21 mol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and are sonicated for 10 min. Prohibitin siRNA is added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 min. to 2 min., and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

When the s.c. tumor is approximately 5.5 mm in size, mice are randomized into treatment groups of 4 including controls and groups receiving taxol or doxorubicin, alone (4 mg/kg), intraperitoneally (i.p.) every 2 days, prohibitin siRNA/liposome formulation alone (3 μg daily for 16 days), or both taxol and prohibitin siRNA/liposome formulation (3 μg daily for 16 days for each treatment). Control siRNA sequences are utilized that do not represent binding sequences to murine prohibitin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the prohibitin siRNA and chemotherapeutic drugs are compared to tumor weights from mice treated with control siRNA and chemotherapeutic drugs.

Mice treated with the prohibitin siRNA have smaller tumors by weight than mice treated with control siRNA. In addition, total cell number in tumors isolated from mice treated with prohibitin siRNA is lower than mice treated with control siRNA.

### Example 11

### Prohibitin Immunoliposome Formulation for Targeted Therapy Against Hematological Cancer Cells

1. Treatment of Hematological Cancer

In order to determine whether targeting prohibitin is useful in treating a preexisting cancerous condition, MHC-matched mice, 5 to 7 weeks old, receive an s.c. injection of the cells 5×10⁵ hematological tumor cells, and tumors are allowed to form. Tumor growth starting on the first day of treatment is measured by palpitation and the volume of the xenograft is monitored every 4 days. Tumors are allowed to grow to a sufficient size (5.5 mm) for appropriate analysis of the effects of prohibitin treatment on tumor sensitivity to chemotherapeutic drugs. Mice are then treated with an immunoliposome formulation containing prohibitin siRNA designed to decrease the level of expression of prohibitin.

Immunoliposome formulations are produced as described by Shi et al. (Proc. Natl. Acad. Sci. USA. (2000) 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and sonicated for 10 min. Prohibitin siRNA is added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

An anti-nucleophosmin mAb is obtained commercially, or is harvested, from serum-free nucleophosmin hybridoma-conditioned media. The anti-nucleophosmin mAb, as well as the isotype control, mouse IgG2a, are purified by protein G Sepharose affinity chromatography. The anti-nucleophosmin mAb or mouse IgG2a (1.5 mg, 10 nmol) is thiolated by using a 40:1 molar excess of 2-iminothiolane (Traut's reagent), as described by Huwyler et al. (Proc. Natl. Acad. Sci. USA. (1996) 93:14164-14169). Thiolated mAb is conjugated to pegylated liposomes using standard procedures also described by Huwyler et al. (Proc. Natl. Acad. Sci. USA. (1996) 93:14164-14169). This preparation is then administered to the animals.

The immunoliposome treatment introduces 3 μg of prohibitin-targeted siRNA per day for 16 days. Control mice receive no treatment, treatment with taxol or doxorubicin alone (4 mg/kg daily) or treatment with liposomes containing control siRNA sequences that are not complementary to murine prohibitin mRNA (3 μg daily for 16 days for each treatment) in combination with taxol or doxorubicin (4 mg/kg daily). Taxol and doxorubicin can be obtained commercially from Sigma Corp. (St. Louis, Mo.).

The cancer cells treated with the immunoliposome/prohibitin siRNA treatment show an increase in sensitivity to chemotherapeutic treatment regimes. As a result, the mice that receive the composition show a better prognosis (i.e., smaller tumor or fewer tumor cells) as compared to mice that receive only the targeting agent or only the vincristin.

A determination of decreased tumor size or cancer cell number is made by sacrificing the mice and excising the tumor. The size of the tumor in mice treated with the prohibitin targeting agent and chemotherapy is measured and compared to measurements obtained from tumors in mice treated with chemotherapy alone. Tumor cell count is determined by trypsinizing tumors in DMEM medium supplemented with 10% fetal bovine serum until cells are in free suspension. Cells are then transferred to six well plates for counting. Cell counts are compared. All experiments are performed in triplicate.

2. Treatment of Adenocarcinoma

In further studies, the efficacy of a prohibitin-targeted therapeutic in treating a mammary adenocarcinoma cells (MCF-7) is assessed. Briefly, male thymic nude mice 5 to 7 weeks old, weighing 18 g to 22 g is used for the MCF-7/ADR xenografts. Mice receive an s.c. injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, prohibitin siRNA/immunoliposome formulation alone (3 μg daily for 16 days), or both taxol and prohibitin siRNA/immunoliposome formulation (3 μg daily for 16 days for each treatment).

Immunoliposome formulations are produced as described by Shi et al. (Proc. Natl. Acad. Sci. USA. (2000) 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and sonicated for 10 min. Prohibitin siRNA is added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

An anti-nucleophosmin mAb is obtained commercially, or is harvested from serum-free nucleophosmin hybridoma-conditioned media. The anti-nucleophosmin mAb, as well as the isotype control, mouse IgG2a, are purified by protein G Sepharose affinity chromatography. The anti-nucleophosmin mAb or mouse IgG2a (1.5 mg, 10 nmol) is thiolated by using a 40:1 molar excess of 2-iminothiolane (Traut's reagent), as described by Huwyler et al. (Proc. Natl. Acad. Sci. USA. (1996) 93:1416-414169). Thiolated mAB is conjugated to pegylated liposomes using standard procedures also described by Huwyler et al. (Proc. Natl. Acad. Sci. USA. (1996) 93:14164-14169). This preparation is then administered to the animals.

Control siRNA sequences are utilized that do not represent binding sequences to murine prohibitin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the prohibitin siRNA and chemotherapeutic drugs are compared to tumor weights from mice treated with control siRNA and chemotherapeutic drugs.

Mice treated with the prohibitin siRNA have smaller tumors by weight than mice treated with control siRNA. In addition, total cell number in tumors isolated from mice treated with prohibitin siRNA is lower than mice treated with control siRNA.

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method of diagnosing chemotherapeutic drug resistance in a neoplastic cell, comprising:
a) detecting a level of prohibitin expressed in a neoplastic cell sample by contacting the cell sample with a probe specific for prohibitin;
b) detecting a level of prohibitin expressed in a non-resistant neoplastic cell control sample of the same tissue type as the neoplastic cell sample by contacting the cell sample with a prohibitin-specific probe; and
c) comparing the level of expressed prohibitin in the neoplastic cell sample to a level of expressed prohibitin in the non-resistant neoplastic cell,
wherein chemotherapeutic drug-resistance is indicated in the neoplastic cell sample if the level of prohibitin expressed in the neoplastic cell sample is greater than the level of prohibitin expressed in the non-resistant neoplastic control cell sample.

2. The method of claim 1, wherein the detection steps comprise isolating a cytoplasmic sample from the neoplastic cell sample and the non-resistant neoplastic control cell sample.

3. The method of claim 1, wherein the prohibitin-targeting agent comprises an anti-prohibitin antibody or a prohibitin binding fragment thereof.

4. The method of claim 3, wherein the level of antibody bound to prohibitin is detected by immunofluorescence, radiolabel, or chemiluminescence.

5. The method of claim 1, wherein the detecting steps comprise hybridizing a nucleic acid probe to a complementary prohibitin mRNA.

6. The method of claim 5, wherein the nucleic acid probe is selected from the group consisting of RNA, DNA, RNA-DNA hybrids, and siRNA.

7. The method of claim 5, wherein the level of nucleic acid probe hybridized to prohibitin mRNA is detected with a label selected from the group consisting of fluorophores, chemical dyes, radiolabels, chemiluminescent compounds, calorimetric enzymatic reactions, chemiluminescent enzymatic reactions, magnetic compounds, and paramagnetic compounds.

8. The method of claim 1, wherein the neoplastic control cell sample is selected from the group consisting of breast adenocarcinoma, breast carcinoma, ovarian carcinoma, and ovarian adenocarcinoma.

9. The method of claim 1, wherein the neoplastic cell sample to be tested is isolated from a mammal.

10. The method of claim 9, wherein the neoplastic cell sample to be tested is isolated from a human.

11. The method of claim 1, wherein the neoplastic cell sample to be tested comprises a breast adenocarcinoma.

12. The method of claim 1, wherein the potentially chemotherapeutic drug-resistant neoplastic cell sample is isolated from a tissue selected from the group consisting of breast, skin, lymphatic, prostate, bone, blood, brain, liver, thymus, kidney, lung, and ovary.

13. A method of treating a neoplasm in a patient in need thereof, comprising:
a) administering an effective amount of a prohibitin-targeting agent to the patient, the targeting agent being capable of binding to prohibitin expressed in the neoplasm; and
b) administering to the patient an effective amount of a chemotherapeutic drug,
wherein the prohibitin targeting agent, when bound to the neoplasm, increases the sensitivity of the neoplasm to the chemotherapeutic drug.

14. The method of claim 13, wherein the prohibitin-targeting agent bound to the neoplasm is internalized into the neoplastic cell.

15. The method of claim 13, wherein the prohibitin-targeting agent comprises a liposome.

16. The method of claim 15, wherein the liposome comprises a neoplastic cell-targeting agent on its surface.

17. The method of claim 13, wherein the prohibitin-targeting agent comprises a nucleic acid.

18. The method of claim 17, wherein the nucleic acid is complementary to a prohibitin mRNA.

19. The method of claim 17, wherein the nucleic acid is selected from the group consisting of RNA, DNA, RNA-DNA hybrids, and siRNA.

20. The method of claim 19, wherein the siRNA comprises 19 contiguous nucleotides of SEQ ID NO: 1.

21. The method of claim 13, wherein the neoplastic cell-targeting agent comprises an antibody, or antigen-binding fragment thereof, specific for a cell marker selected from the group consisting of multidrug resistance protein 1, BRCP, p53, vimentin, α-enolase, nucleophosmin, and HSC70.

22. The method of claim 13, wherein the prohibitin-targeting agent is administered to the patient by injection at the site of the neoplasm.

23. The method of claim 13, wherein the prohibitin-targeting agent is administered to the patient by surgical introduction at the site of the neoplasm.

24. The method of claim 13, wherein the prohibitin-targeting agent is administered to the patient by inhalation of an aerosol or vapor.

25. The method of claim 13, wherein the neoplasm to be treated is chemotherapeutic drug-resistant.

26. The method of claim 13, wherein the chemotherapeutic drug is selected from the group consisting of Daunorubicin, Docetaxel, Doxorubicin, Etoposide, Idarubicin, Melphalan, Mitoxantrone, Paclitaxel, Taxol, Teniposide, Topotecan, Vinblastine, Vincristin, and combinations thereof.

27. A kit for detecting chemotherapeutic drug resistance in a neoplastic cell sample, comprising:
a) a first probe for the detection of prohibiting
b) a second probe for the detection of chemotherapeutic drug resistance, the second probe being specific for a marker selected from the group consisting of multidrug resistance protein 1, BRCP, p53, vimentin, α-enolase, nucleophosmin, and HSC70; and
c) detection means for identifying probe binding to a target.

28. The kit of claim 27, wherein the first probe is a nucleic acid that is complementary to mRNA encoding prohibitin.

29. The kit of claim 28, wherein the nucleic acid is selected from the group consisting of RNA, DNA, RNA-DNA hybrids, and siRNA.

30. The kit of claim 27, wherein the second probe comprises a nucleic acid complementary to an mRNA encoding multidrug resistance protein 1, BRCP, p53, vimentin, α-enolase, nucleophosmin, or HSC70.

31. The kit of claim 30, wherein the nucleic acid probe is selected from the group consisting of RNA, DNA, RNA-DNA hybrids, and siRNA.

32. The kit of claim 27, wherein the second probe comprises an antibody or prohibitin binding fragment thereof.

33. The kit of claim 27, wherein the detection means is selected from the group consisting of fluorophores, chemical dyes, radiolabels, chemiluminescent compounds, colorimetric enzymatic reactions, chemiluminescent enzymatic reactions, magnetic compounds, and paramagnetic compounds.

34. A pharmaceutical formulation for treating a neoplasm, comprising:
a) a prohibitin-targeting component;
b) a chemotherapeutic drug; and
c) a pharmaceutically acceptable carrier.

35. The pharmaceutical formulation of claim 34, wherein the prohibitin-targeting component is a nucleic acid.

36. The pharmaceutical formulation of claim 35, wherein the nucleic acid is selected from the group consisting of RNA, DNA, RNA-DNA hybrids, and siRNA.

37. The pharmaceutical formulation of claim 36, wherein the prohibitin-targeting component is a siRNA.

38. The pharmaceutical formulation of claim 37, wherein the siRNA has a GC content of at least 40%.

39. The pharmaceutical formulation of claim 37, wherein the siRNA comprises 19 contiguous nucleotides of SEQ ID NO: 1.

40. The pharmaceutical formulation of claim 34, wherein the prohibitin-targeting agent comprises an antibody or prohibitin-binding fragment thereof.

41. The pharmaceutical formulation of claim 34, wherein the prohibitin-targeting agent comprises a liposome.

42. The pharmaceutical formulation of claim 42, wherein the liposome comprises a neoplastic cell-targeting agent on its surface.

43. The pharmaceutical formulation of claim 43, wherein the neoplastic cell-targeting agent is an antibody, or binding fragment thereof.

44. The pharmaceutical formulation of claim 44, wherein the neoplastic cell-targeting agent binds to a neoplastic cell marker selected from the group consisting of multidrug resistance protein 1, BRCP, p53, vimentin, α-enolase, nucleophosmin, and HSC70.

45. The pharmaceutical formulation of claim 34, wherein the chemotherapeutic drug is selected from the group consisting of Daunorubicin, Docetaxel, Doxorubicin, Etoposide, Idarubicin, Melphalan, Mitoxantrone, Paclitaxel, Taxol, Teniposide, Topotecan, Vinblastine, Vincristin, and combinations thereof.


SEQUENCE LISTING (4 sequences)

SEQ ID NO: 1 - RNA, 19 nt, Artificial Sequence
  Description of Artificial Sequence Synthetic oligonucleotide
uaacagacagaccacuucc

SEQ ID NO: 2 - RNA, 19 nt, Artificial Sequence
  Description of Artificial Sequence Synthetic oligonucleotide
ggaaguggucugucuguua

SEQ ID NO: 3 - DNA, 21 bp, Artificial Sequence
  Description of Combined DNA/RNA Molecule Synthetic oligonucleotide
  Description of Artificial Sequence Synthetic oligonucleotide
ggaaguggucugucuguuatt

SEQ ID NO: 4 - DNA, 21 bp, Artificial Sequence
  Description of Combined DNA/RNA Molecule Synthetic oligonucleotide
  Description of Artificial Sequence Synthetic oligonucleotide
uaacagacagaccacuucctt